SEQUENCE LISTING (7 sequences)

SEQ ID NO: 1 - protein, 20 aa, Artificial sequence
  Synthetic peptide
LKKFNARRKLKGAILTTMLA

SEQ ID NO: 2 - protein, 21 aa, Artificial sequence
  Synthetic peptide
MHRQEAVDCLKKFNARRKLKG

SEQ ID NO: 3 - protein, 29 aa, Artificial sequence
  Synthetic peptide
MHRQETVDCLKKFNARRKLKGAILTTMLA

SEQ ID NO: 4 - DNA, 20 bp, Artificial sequence
  Synthetic oligonucleotide
ctgtaccagcagatcaaagc

SEQ ID NO: 5 - DNA, 20 bp, Artificial sequence
  Synthetic oligonucleotide
atcactagcaccatgtggtc

SEQ ID NO: 6 - DNA, 21 bp, Artificial sequence
  Synthetic oligonucleotide
caccaccauugaggacgaatt

SEQ ID NO: 7 - DNA, 21 bp, Artificial sequence
  Synthetic oligonucleotide
ttguggugguaacuccugcuu

(19) United States
(12) Patent Application Publication
(10) Pub. No.: US 2016/0193222 A1
(43) Pub. Date: Jul. 7, 2016

(54) METHOD FOR TREATING PAIN WITH A CALMODULIN INHIBITOR

(71) Applicant: The Board of Trustees of the University of Illinois, Urbana, IL (US)

(72) Inventor: Zaijie Wang, Oak Park, IL (US)

(21) Appl. No.: 15/056,338

(22) Filed: Feb. 29, 2016

Related U.S. Application Data
(63) Continuation of application No. 12/646,074, filed on Dec. 23, 2009.
Application No. 12/646,074 is a continuation-in-part of application No. 11/768,586, filed on Jun. 26, 2007, now abandoned.
Application No. 11/768,586 is a continuation-in-part of application No. 10/769,536, filed on Jan. 30, 2004, now Pat. No. 7,256,200.
(60) Provisional application No. 60/446,232, filed on Feb. 10, 2003.
(60) Provisional application No. 60/806,002, filed on Jun. 28, 2006.
(60) Provisional application No. 60/897,979, filed on Jan. 29, 2007.

(51) Int. Cl. A61K 31/5415; A61K 45/06

(52) U.S. Cl. CPC A61K 31/5415; A61K 45/06

(57) ABSTRACT

The present invention relates to the use of Ca²⁺/CaM-dependent protein kinase II (CaMKII) inhibitors alone and in combination with opiate analgesics for treating pain, in particular chronic pain. Methods for reducing or reversing tolerance, dependence, and opioid-induced hyperalgesia are also provided.

### INTRODUCTION

This application is a continuation of U.S. patent application Ser. No. 12/646,074, filed Dec. 23, 2009, which is a continuation-in-part application of U.S. patent application Ser. No. 11/768,586, filed Jun. 26, 2007, which is a continuation-in-part application of U.S. patent application Ser. No. 10/769,536, filed Jan. 30, 2004, now issued as U.S. Pat. No. 7,256,200, which claims benefit of U.S. Provisional Patent Application Ser. No. 60/897,979, filed Jan. 29, 2007; U.S. Provisional Patent Application Ser. No. 60/806,002, filed Jun. 28, 2006; and U.S. Provisional Patent Application Ser. No. 60/446,232, filed Feb. 10, 2003; the contents of which are incorporated herein by reference in their entireties.

This invention was made in the course of research sponsored by the National Institutes of Health (NIH grant Nos. DA005050, HL098141, and AT003647). The U.S. government has certain rights in this invention.

### BACKGROUND OF THE INVENTION

One of the most significant health problems is an inadequate control of pain, especially chronic pain associated with diseases such as cancer, back pain, arthritis, and diabetic neuropathy. It is estimated that the annual cost for health care and lost productivity related to pain is over $100 billion in the U.S. However, the impact of pain on society is measured not only in economic numbers, but, more importantly, by suffering. For example, more than 50 million Americans are partially or totally disabled by chronic pain, which accounts for about one-fourth of all workdays lost annually.

Analgesics are agents that relieve pain by acting centrally to elevate pain threshold, preferably without disturbing consciousness or altering other sensory functions. A mechanism by which analgesic drugs obtund pain (i.e., raise the pain threshold) has been formulated. Research in this area has resulted in the development of a number of opiate and opioid analgesics having diverse pharmacological actions. While opioid analgesics remain the mainstay for pain treatment, prolonged use of these drugs leads to tolerance that results in frequent dose escalation and increased side effects, such as altered cognitive state and inadequate pain control, and possibly drug dependence.

Effective pain therapies directed to preventing opioid tolerance have long been sought. The success of developing such effective therapies requires a better understanding of the underlying tolerance mechanisms. Opioid receptor internalization, down-regulation, and uncoupling from G proteins (desensitization) all have been proposed as potential mechanisms. However, no consistent changes have been identified (Nestler (1994) Neuropsychopharmacology 11:77-87; Nestler, et al. (1997) Science 278:58-63). A phenomena called “cAMP upregulation” has been proposed as a biochemical correlation for opioid tolerance (Sharma, et al. (1975) Proc. Natl. Acad. Sci. USA 72:3092-3096; Wang, et al. (1994) Life Sci. 54:L339-350; Nestler (1994) supra). This theory was expanded when linked to the regulation of protein kinase A (PKA) and CREB activation in cellular model of opioid tolerance (Nestler (1994) supra; Nestler (1997) Curr. Opin. Neurobiol. 7:713-719). However, studies with CREB mutant mice suggested that CREB may be a factor more important for opioid dependence (Maldonado, et al. (1996) Science 273:657-659; Blendy, et al. (1998) J. Mol. Med. 76:104-110). Inhibition of PKA has produced an inconsistent effect on behavioral manifestations of opioid tolerance (e.g., Narita, et al. (1995) Eur. J. Pharmacol. 280:R1-3; Bilsky, et al. (1996) J. Pharmacol. Exp. Ther. 277:484-490; Shen, et al. (2000) Synapse 38:322-327).

Other studies found that NMDA receptor antagonists were involved in the development of opioid tolerance (Mao, et al. (1995) Pain 61:353-364). Central to these findings is increased intracellular Ca²⁺ levels resulting from NMDA receptor activation and other neuronal activation. In this regard, the use of agents which modulate NMDA receptors to treat pain has been suggested (see, e.g., U.S. Pat. Nos. 5,502,058 and 6,406,716). Calcium ion (Ca²⁺) is used as a second messenger in neurons, leading to the activation various protein kinases, among them, Ca²⁺/phospholipids-dependent protein kinase (PKC) and Ca²⁺/calmodulin-dependent protein kinase II (CaMKII). PKC has been implicated in opioid tolerance (Coderre, et al. (1994) Eur. J. Neurosci. 6:1328-1334; Mao, et al. (1995) supra; Granados-Soto, et al. (2000) Pain 85:395-404; Narita, et al. (2001) Pharmacol. Ther. 89(1):1-15). Mice lacking PKC exhibited significantly reduced opioid tolerance (Zeitz, et al. (2001) Pain 94:245-253). NMDA receptors are known to interact with CaMKII by Ca²⁺ influx and phosphorylation. It is unclear from these studies, however, whether CaMKII plays a role in the development and/or maintenance of opioid tolerance.

CaMKII is a multifunctional calcium and calmodulin activated kinase, whose α and β isoforms are abundant in the central nervous system. A vast amount of information is available for the interaction of CaMKII α isoform and NMDA receptor in long-term potentiation in hippocampal neurons, which is critical for learning and memory (e.g., Mayford, et al. (1996) Science 274:1678-1683). Glutamate can activate CaMKII through NMDA receptor and Ca²⁺ influx in cultured rat hippocampal neurons (Fukunaga, et al. (1992) J. Biol. Chem. 267:22527-22533). Calcium influx via NMDA receptors results in activation and Thr286 autophosphorylation of CaMKII (Strack, et al. (1998) J. Biol. Chem. 273:20689-20692; Strack, et al. (2000) J. Biol. Chem. 275:23798-23806). On the other hand, CaMKII phosphorylates and activates the NMDA receptor, and enhances Ca²⁺ influx through the channel (Kitamura, et al. (1993) J. Neurochem. 61:100-109).

No direct information exists for the role of CaMKII or NMDA/CaMKII interaction in opioid tolerance. Indirectly, chronic opioid administration increases both the level (Lou, et al. (1999) Mol. Pharmacol. 55:557-563) and activity (Nehmad, et al. (1982) Mol. Pharmacol. 22:389-394) of calmodulin, as well as calmodulin mRNA levels (Niu, et al. (2000) Jpn. J. Pharmacol. 84:412-417). Cytosolic-free Ca²⁺ also can be increased after treatment with opioids (Fields, et al. (1997) Life Sci. 61:595-602; Quillan, et al. (2002) J. Pharmacol. Exp. Ther. 302:1002-1012). CaMKII also has been shown to phosphorylate and activate the cAMP response element binding protein (CREB) (Wu & McMurry (2001) J. Biol. Chem. 276(3):1735-41). More direct evidence arose from the finding that CaMKII and μ opioid receptor (μOR) are colocalized in the superficial layers of the spinal cord dorsal horn, an area critical for pain transmission (Bruggemann, et al. (2000) Brain Res. Mol. Brain Res. 85:239-250). The cloned μOR contains several consensus sites for phosphorylation by CaMKII (Mestek, et al. (1995) J. Neurosci. 15:2396-2406). Desensitization of μOR was enhanced when CaMKII was overexpressed (Mestek, et al. (1995) supra; Koch, et al. (1997) J. Neurochem. 69:1767-1770). Recently, hippocampal, but not striatal, CaMKII was found to modulate opioid tolerance and dependence by affecting memory pathways (Fan, et al. (1999) Mol. Pharmacol. 56:39-45; Lou, et al. (1999) supra). Moreover, spinal CaMKIIα was activated in capsaicin-induced inflammation (Fang, et al. (2002) J. Neurosci. 22:4196-4204). The role of spinal CaMKII in opioid tolerance was not discussed.

Data has suggested that Ca²⁺-mediated cell signaling is important in nociception (Ben-Sreti, et al. (1983) Eur. J. Pharmacol. 90:385-91; Kim, et al. (2003) Science 302:117-9; Saegusa, et al. (2001) EMBO J. 20:2349-56; Spampinato, et al. (1994) Eur. J. Pharmacol. 254:229-38; White & Cousins (1998) Brain Res. 801:50-8). However, while the levels of CaMKII and phosphorylated CaMKII (pCaMKII) have been shown to be significantly increased in the spinal cord within minutes after an intradermal injection of capsaicin (Fang, et al. (2002) J. Neurosci. 22:4196-204), it has not been previously demonstrated that the CaMKII signaling pathway modulates pain.

### SUMMARY OF THE INVENTION

The present invention is a method for preventing or treating pain by administering to a subject in need of treatment an effective amount of a calcium calmodulin-dependent protein kinase II (CaMKII) inhibitor. In some embodiments, the CaMKII inhibitor is a calcium blocker, a calcium chelator, a CaMKII antagonist, a small peptide based on CaMKII protein sequence, a nucleic acid-based inhibitor such as an siRNA molecules (e.g. SEQ ID NOs:6 and 7), or a mixture thereof. In other embodiments, the pain is acute or chronic pain, wherein chronic pain includes cancer pain, post-traumatic pain, post-operative pain, neuropathic pain, inflammatory pain or pain associated with a myocardial infarction. In particular embodiments, the CaMKII inhibitor is administered simultaneously or sequentially with an effective amount of an opiate analgesic. In accordance with such embodiments, the opiate analgesic is an opium alkaloid, a semisynthetic opiate analgesic, or a mixture thereof.

The present invention also provides methods for reducing, reversing, or preventing tolerance to an opiate analgesic, dependence on an opiate analgesic in a subject undergoing opiate analgesic therapy, and opioid-induced hyperalgesia in a subject by administrating to the subject an effective amount of a CaMKII inhibitor. A method for treating opiate analgesic withdrawal with a CaMKII inhibitor is also provided.

### BRIEF DESCRIPTION OF THE DRAWINGS

FIGS. 1A and 1B show the prevention of complete Freund's adjuvant (CFA)-induced thermal hyperalgesia and mechanical allodynia by the Ca²⁺/calmodulin-dependent protein kinase II (CaMKII) inhibitor KN93. CFA intraplantar injection induced mechanical allodynia (FIG. 1A) and thermal hyperalgesia (FIG. 1B) in mice. Pre-treatment with KN93 (30 nmol, i.t.) followed by two additional doses on Day 1 and Day 3 prevented the development of thermal hyperalgesia and mechanical allodynia. Data are expressed in Mean±SEM. *p<0.05 compared with the control group; # p<0.05 compared with the CFA group. Arrows indicate the time (30 minutes before behavior test) when KN93 or normal saline was injected.

FIG. 2 shows that the CaMKII inhibitor KN93 suppressed the increased CaMKII activation (pCaMKII) in the lumbar spinal cord in CFA mice. Representative western immunoblots of lumbar spinal cord were subjected to densitometric analysis. Optical density (OD) of pCaMKII was normalized to β-actin. Treatments were: CFA/KN92, CFA+KN92 nmol, i.t.; CFA/KN93 (30), CFA+KN93 30 nmol, i.t.; CFA/KN93 (45), CFA+KN93 45 nmol, i.t.; KN93 (30)/CFA, KN93 nmol, i.t. 1 hour before CFA injection. Data are expressed in Mean±SEM. *p<0.05 compared with the control group; # p<0.05 compared with the CFA group.

FIGS. 3A and 3B show the reversal of CFA-induced thermal hyperalgesia and mechanical allodynia by the CaMKII inhibitor KN93. CFA intraplantar injection induced mechanical allodynia (FIG. 3A) and thermal hyperalgesia (FIG. 3B) in mice. KN93 (45 nmol, i.t.), but not 30 nmol, administered at 24 hours and 72 hours after CFA injection reversed these pain behaviors. KN92 (45 nmol, i.t.) did not have any effect. Data are expressed in Mean±SEM. *p<0.05 compared with the control group; # p<0.05 compared with the CFA group. Arrows indicate the time (30 minutes before behavior test) when KN93, KN92, or normal saline was injected.

FIGS. 4A and 4B show the reversal of spinal nerve ligation (SNL)-induced thermal hyperalgesia and mechanical allodynia by the CaMKII inhibitor KN93. L5/L6 spinal nerve ligation induced mechanical allodynia (FIG. 4A) and thermal hyperalgesia (FIG. 4B) in mice. Post-treatment with KN93 (45 nmol, i.t) on Day 5 after SNL operation reversed thermal hyperalgesia and mechanical allodynia. KN92 (45 nmol, i.t.) had no effect. Sham, sham operation; SNL, L5/L6 spinal nerve ligation; SNL/KN92 (45), SNL+KN92 45 nmol, i.t.; SNL/KN93 (45), SNL+KN93 45 nmol, i.t. An arrow indicates a single dose of KN93 was given 30 minutes before behavior test on Day 5. Data are expressed in Mean±SEM. *P<0.05 compared with Sham operation group; **p<0.01 compared with the Sham operation group; # p<0.05 compared with SNL group.

FIG. 5 shows the CaMKII inhibitor KN93 suppressed the increased CaMKII activation (pCaMKII) in lumbar spinal cord in SNL mice. Representative western immunoblots of lumbar spinal cord were subjected to densitometric analysis. Optical density (OD) of pCaMKII was normalized to β-actin. Sham, sham operation; SNL, L5/L6 spinal nerve ligation; SNL/KN92 (45), SNL+KN92 45 nmol, i.t.; SNL/KN93 (45), SNL+KN93 45 nmol, i.t. Data are expressed in Mean±SEM. *p<0.05 compared with the sham operation group; # p<0.05 compared with the SNL group.

FIGS. 6A and 6B show the reversal of CFA-induced thermal hyperalgesia and mechanical allodynia by the CaMKII inhibitor Trifluoperazine. CFA intraplantar injection induced mechanical allodynia (FIG. 6A) and thermal hyperalgesia (FIG. 6B) in mice. Trifluoperazine (0.5 mg/kg, i.p.) administered at 24 hours after CFA injection reversed these pain behaviors. Low dose (0.25 mg/kg, i.p.) of Trifluoperazine slightly alleviated mechanical allodynia. Baseline, before CFA injection; post-induction, 1 day after CFA injection; post-treatment, 30 minutes after TFP; or saline intraperitoneal injection. Data are expressed in Mean±SEM. *p<0.05, **p<0.01, ***p<0.001 compared with the control group; # p<0.05, ### p<0.001 compared with the CFA group.

FIGS. 7A and 7B show the reversal of SNL-induced thermal hyperalgesia and mechanical allodynia by the CaMKII inhibitor Trifluoperazine. L5/L6 spinal nerve ligation induced mechanical allodynia (FIG. 7A) and thermal hyperalgesia (FIG. 7B) in mice. Trifluoperazine (0.5 mg/kg, i.p.) administered 5 days after SNL reversed these pain behaviors. Low dose (0.25 mg/kg, i.p.) of Trifluoperazine slightly increase nociceptive threshold. Baseline, before SNL; Post-operation, 5 days after SNL or sham operation; Post-treatment, 30 minutes after TFP or saline injection. Data are expressed in Mean±SEM. *p<0.05, **p<0.01, ***p<0.001 compared with the control group; # p<0.05, ## p<0.01, ### p<0.001 compared with the SNL group.

FIG. 8 is a bar graph showing the percent of maximal possible effect (MPE %) of placebo, morphine treated (MS), and morphine/KN93 treated groups.

FIGS. 9A, 9B and 9C show the effect of CaMKII inhibition on opioid tolerance. FIG. 9A shows that CaMKII inhibition dose-dependently reverses established opioid tolerance, whereas, FIG. 9B shows that CaMKII inhibition prevents opioid tolerance. MPE % is percent of maximal possible effect. FIG. 9C shows that CaMKII inhibition prevents opioid dependence. PB is placebo, MS is morphine sulfate.

FIGS. 10A and 10B contain bar graphs respectively showing that CaMKII inhibition reverses established opioid tolerance and reverses established opioid dependence. MPE % is percent of maximal possible effect, MS is morphine sulfate.

FIG. 11 shows the effect of trifluoperazine on basal nociception and acute morphine antinociception. To test the effect of trifluoperazine on basal thermal nociception, mice were treated with trifluoperazine (0.5 mg/kg, i.p. “Tri”) or normal saline (“NS”) 30 minutes before the tail-flick test. To investigate the effect of trifluoperazine on acute morphine antinociception, trifluoperazine (0.5 mg/kg, i.p., “Tri/MS(dose)” group) or normal saline (“NS/MS(dose)” group) was given to mice 30 minutes before the test dose of morphine (3 or 10 mg/kg, s.c.). Results are presented in “% MPE” as defined as “100×(test-control)/(cut-off-control)”, and expressed as mean±S.E.M (n 6 for each group). Trifluoperazine produced slight antinociception by itself, but did not affect morphine induced antinociception (p>0.05). ***p<0.001 compared with the normal saline group, Student's t-test.

FIG. 12 shows the effect of trifluoperazine on antinociceptive tolerance to morphine. Male ICR mice were injected with morphine (100 mg/kg, s.c.) to induce tolerance. Control mice received the same volume of normal saline. Five hours later, the antinociception produced by a test dose of morphine (10 mg/kg, s.c.) was evaluated using a 52° C. warm-water tail-flick assay. A cut-off of 12 seconds was used to prevent tissue damage. Data are expressed as mean±S.E.M (n=8 for each group). Morphine (100 mg/kg, s.c.) induced opioid antinociceptive tolerance (“MS” group), as evidenced by the significantly decreased antinociception. Trifluoperazine (0.5 mg/kg, i.p.) given 30 minutes before the test dose of morphine (“MS+acute Tri” group) was able to reverse the established tolerance. Mice copretreated with trifluoperazine (0.5 mg/kg, i.p.) and morphine (100 mg/kg, s.c.) (“co-pretreatment w/Tri+MS” group) developed significantly less tolerance. *p<0.05, ***p<0.001 compared with the normal saline group; ###p<0.001 compared with the “MS” group, Student's t-test.

FIGS. 13A and 13B show the effect of trifluoperazine on superspinal (FIG. 13A) and spinal (FIG. 13B) CaMKII activity. Solubilized brain and spinal tissue samples were subjected to 10% polyacrylamide gel electrophoresis and transferred onto PVDF membranes, which were then incubated with anti-pCaMKII and HRP-conjugate anti-rabbit secondary antibody. Ratios of the optical densities of pCaMKII to that of β-actin were calculated for each sample. Data are expressed as mean±S.E.M (n=3 for each group). Both supraspinal and spinal CaMKII activity was significantly up-regulated in tolerant mice (“MS” group), which was reduced by the pretreatment (“co-pretreatment w/Tri+MS” group) or acute treatment (“MS+acute Tri” group) with trifluoperazine. *p<0.05, ***p<0.001 compared with the “saline” group; ##p<0.01, ###p<0.001 compared with the “MS” group, Student's t-test.

FIG. 14 shows the effect of the acute treatment of haloperidol on acute morphine tolerance. Groups of six ICR male mice received morphine (100 mg/kg s.c.) or an equal volume of saline (Saline). Four hours later haloperidol (0.06, 0.20, 0.60 mg/kg i.p.) was given to several groups and the remaining groups received an i.p. injection of saline. Half hour later, all groups received a test dose of morphine (10 mg/kg s.c.) and antinociception was determined by the tail-flick assay 30 minutes after. Data are expressed in % MPE (mean±S.E.M.). *, p<0.05; ***, p<0.001 compared with the Saline group; ##, p<0.01; ###, p<0.001 compared with 0.00 MS/haloperidol group.

FIG. 15 shows the effect of haloperidol on basal antinociception and morphine antinociception. Groups of six ICR male mice were given haloperidol (2 mg/kg i.p.) or equal volume of saline (NS, MS). Thirty minutes later low dose of morphine (3 mg/kg s.c.) was given to MS and Halo+MS groups. The remaining groups received equal volume of saline. The antinociception effects were determined by tail-flick assay half hour later for all four groups. Data are expressed in % MPE (mean±S.E.M.). ***, p<0.001 compared with the Saline group; ###, p<0.001 compared with the Halo group.

FIG. 16 shows the effect of the acute treatment of haloperidol on acute morphine dependence. Groups of six ICR male mice received morphine (100 mg/kg s.c.) or an equal volume of saline (Saline). Four hours later haloperidol (0.06, 0.20, 0.60 mg/kg i.p.) was given to several groups and the remaining groups received an i.p. injection of saline. Half hour later, all groups received a test dose of morphine (10 mg/kg s.c.). Development of morphine dependence, as revealed by 10 mg/kg i.p. naloxone-precipitated withdraw jumping (30 minutes after the test dose of morphine) was reversed by haloperidol in a dose dependent manner. Data are expressed as mean±S.E.M. **, p<0.01; ***, p<0.001 compared with the Saline group; ##, p<0.01; ###, p<0.001 compared with 0.00 MS/haloperidol group.

FIG. 17 shows the prevention of acute opioid tolerance by haloperidol. Groups of six male ICR male mice received haloperidol (0.20, 0.60, 1.00 mg/kg i.p.) or equal volume of saline (0.00 haloperidol/MS) immediately prior to administration of morphine (100 mg/kg s.c.). Saline mice only received s.c. injection of saline. Four hours later all groups received a test dose of morphine (10 mg/kg s.c.). The antinociception was determined by the tail-flick assay 30 minutes after. Data are expressed in % MPE (mean±S.E.M.). ***, p<0.001 compared with the Saline group; #, p<0.05; ###, p<0.001 compared with 0.00 haloperidol/MS group.

FIG. 18 shows the prevention of acute opioid dependence by haloperidol. Groups of six male ICR male mice received haloperidol (0.20, 0.60, 1.00 mg/kg i.p.) or equal volume of saline (0.00 haloperidol/MS) immediately prior to administration of morphine (100 mg/kg s.c.). Saline mice only received s.c. injection of saline. Four hours later all groups received a test dose of morphine (10 mg/kg s.c.). Development of morphine dependence, as revealed by 10 mg/kg i.p. naloxone-precipitated withdraw jumping (30 minutes after the test dose of morphine) was prevented by haloperidol in a dose dependence way. Data are expressed as mean±S.E.M. **, p<0.01; ***, p<0.001 compared with the Saline group; ###, p<0.001 compared with 0.00 haloperidol/MS group.

FIGS. 19A and 19B show the effect of haloperidol on brain and spinal cord pCaMKII activity in opioid tolerance and dependence mice. Groups of three ICR male mice received morphine (100 mg/kg s.c.) or an equal volume of saline (Saline). Four hours later haloperidol (0.60, 0.20, 0.06 mg/kg i.p.) was given to MS/halo groups and the remaining groups received an i.p. injection of saline. For halo/MS group, the mice received haloperidol (0.6 mg/kg i.p.) immediately prior to administration of morphine. Brain (FIG. 19A) and spinal cord (FIG. 19B) samples of each group were taken four and a half hours of morphine or saline injection to determine the CaMKII activity. The activated CaMKII was determined by the western blot analysis using an antibody specific for Thr286-pCaMKII. Histogram data, expressed as mean±S.E.M., were constructed from four representative experiments. *, p<0.05; **, p<0.01 compared with the Saline group; #, p<0.05 compared with 0.00 MS/haloperidol group.

FIGS. 20A and 20B show the reversal of morphine-induced mechanical allodynia (FIG. 20A) and thermal hyperalgesia (FIG. 20B) by KN93. Opioid-induced hyperalgesia (OIH) was induced by intermittent morphine injections. On day 5, mice received KN93 (15-45 nmol, i.t.), KN92 (45 nmol, i.t.), or saline (i.t.) at time 0. Mechanical allodynia and thermal hyperalgesia were tested at the different time points as indicated. KN93, but not KN92, reversed the established morphine-induced mechanical allodynia and thermal hyperalgesia in a dose- and time-dependent manner. Data are expressed in Mean±SEM; n=8 for each group.

FIGS. 21A and 21B show the suppression of morphine-induced CaMKIIα activation by KN93. Morphine or saline treated mice were administered (i.t.) with KN93 (15-45 nmol), KN92 (45 nmol), or saline on day 5. One hour later, mice were sacrificed and the lumbar spinal cords were taken for the analysis of CaMKIIα activation using the immunoblotting method, by determining the degree of CaMKIIα autophosphorylation (pCaMKIIα). Morphine enhanced pCaMKIIα expression, without altering CaMKIIα expression. KN93, but not its inactive analog, KN92, reversed morphine enhanced CaMKIIα activation. Data are expressed in Mean±SEM. *p<0.05, compared with the saline treated group; † p<0.05, compared with the morphine treated group; n=4 for each group.

FIGS. 22A and 22B show the reversal of OIH by siRNA-mediated CaMKIIα knockdown. In phase 1, mice received repeated saline or morphine administration. Mechanical and thermal sensitivities were measured on day 5. In phase 2, after OIH had fully developed, mice were treated with CaMKIIα siRNA or scrambled siRNA (2 μg, twice/day for 3 days). Mechanical and thermal sensitivities were tested daily. Established OIH was reversed by CaMKIIα siRNA. Data are expressed in Mean±SEM. ***p<0.001, compared with the saline treated group; ††† p<0.001, compared with the morphine treated group; n=5 for each group. Arrows indicated saline, CaMKIIα or scrambled siRNA administration.

### DETAILED DESCRIPTION OF THE INVENTION

It has now been found that the Ca2+/calmodulin-dependent protein kinase II (CaMKII) signaling pathway is involved in the process of chronic pain and opioid tolerance and dependence. Pretreatment with CaMKII inhibitors prevents development of thermal hyperalgesia, opioid-induced hyperalgesia and mechanical allodynia and post-treatment with CaMKII inhibitors completely reverses these pain behaviors. Moreover, administration of a CaMKII inhibitor with morphine restores the effectiveness of morphine in animals that are tolerant to even very large doses of morphine. Accordingly, the CaMKII signaling pathway now provides a novel target for treating chronic pain and preventing or reversing opioid tolerance and dependence.

Autophosphorylation and activation of CaMKII are molecular mechanisms that have been proposed to contribute to long-term potentiation in the hippocampus (Malinow, et al. (1988) Nature 335(6193):820-4; Mayford, et al. (1995) Cell 81(6):891-904; Fukunaga, et al. (1996) Neurochem. Int. 28(4):343-58; Soderling & Derkach (2000) Trends Neurosci. 23(2):75-80). Spinal CaMKII has been suggested to contribute to noxious stimulation-evoked central sensitization in a manner similar to its role in the processes underlying long-term potentiation (Fang, et al. (2002) supra). It has also been suggested that CaMKII contributes to two models of neuropathic pain induced by chronic constriction injury (CCI) of sciatic nerve (Dai, et al. (2005) Eur. J. Neurosci. 21(9):2467-74) or inferior alveolar nerve (IAN) transaction (Ogawa, et al. (2005) Exp. Neurol. 192(2):310-9).

It has now been found that CaMKII is important in the maintenance as well as generation of chronic pain. This is in contrast to the teachings of the prior art which discloses only pre- but not post-treatment with KN93 can attenuate the development of thermal hyperalgesia and mechanical allodynia in CCI rats (Dai, et al. (2005) supra). The differences in these findings may be the result of different KN93 dosages; a 45 nmol dosage was employed herein, whereas the art teaches a 10 nmol/day dose. Treatment or reversal of pain was an unexpected result because once CaMKII is autophosphorylated on Thr286, its activity is no longer dependent on calcium-calmodulin (Kennedy, et al. (1990) Cold Spring Harb. Symp. Quant. Biol. 55:101-10; Lisman (1994) Trends Neurosci. 17(10):406-12) and therefore its activity may not have been affected by KN93 application. However, because the results indicate that both pre- and post-treatment with KN93 could equally block the increase in pCaMKII, it is believed that KN93 inhibited the ongoing, continuous phosphorylation of pCaMKII, which constitutes the main portion of total pCaMKII. This may also explain why post-treatment with low dosage of KN93, such as 30 nmol in CFA model, could not block the increase in pCaMKII, and thus could not reverse pain behavior.

Having demonstrated the critical involvement of CaMKII in the maintenance of chronic pain and the use of CaMKII inhibitors to prevent and treat pain alone or in combination with an opioid analgesic, the present invention is a method for preventing or treating pain by administering to a subject in need of treatment a CaMKII inhibitor. As used in the context of the present invention, a CaMKII inhibitor includes any compound which interacts with calmodulin; and/or operates on the catalytic and regulatory, linker, association, and other domains of CaMKII; and/or selectively inhibits the enzymes activated by calcium and/or calmodulin thereby treating pain in a subject. Accordingly, CaMKII inhibitors of the present invention include a calcium blocker or chelator, a CaMKII antagonist, a small peptide based on CaMKII protein sequence, a nucleic acid-based inhibitor, or a mixture thereof

Calcium blockers and chelators include compounds which control calcium channel activity, i.e., channels actuated by the depolarization of cell membranes thereby allowing calcium ions to flow into the cells. Such compounds inhibit the release of calcium ions from intracellular calcium storage thereby blocking signaling through the CaMKII pathway. Exemplary calcium blockers include, e.g., 1,4-dihydropyridine derivatives such as nifedipine, nicardipine, niludipine, nimodipine, nisoldipine, nitrendipine, milbadipine, dazodipine, and ferodipine; N-methyl-N-homoveratrilamine derivatives such as verapamil, gallopamil, and tiapamil; benzothiazepine derivatives such as diltiazem; piperazine derivatives such as cinnarizine, lidoflazine, and flunarizine; diphenylpropiramine derivatives such as prenylamine, terodiline, and phendiline; bepridil; and perhexyline. Exemplary calcium chelators include, e.g., BAPTA tetrasodium salt, 5,5′-Dibromo-BAPTA tetrasodium salt, BAPTA/AM, 5,5′-Difluoro-BAPTA/AM, EDTA tetrasodium salt (Ethylenediamine tetraacetic acid), EGTA (Ethylenebis(oxyethylenenitrilo)tetraacetic acid), EGTA/AM, MAPTAM, and TPEN.

CaMKII antagonists include inhibitors that operate on the catalytic, regulatory, linker, or association domains of CaMKII. Exemplary CaMKII antagonists include known small molecule CaMKII inhibitors such as KN62 (Kamiya Biomedical, Thousand Oaks, Calif.), KN93, H89, HA1004, HA1077, autocamtide-2 related inhibitory peptide (AIP), K-252a, Staurosporine, Lavendustin C; anti-psychotic CaMKII inhibitors including, e.g., phenothiazines such as chlorpromazine, fluphenazine, mesoridazine, perphenazine, pipotiazine, prochlorperazine, promazine, thioproperazine, thioridazine, trifluoperazine, triflupromazine, chlorprothixene, clozapine, haloperidol, pimozide, and promethazine; calmodulin antagonists such as calmidazolium chloride, calmodulin binding domain, chlorpromazine, compound 48/80, melittin, ophiobolin A, pentamidine isethionate, phenoxybenzamine, W-5, W-7, W-12, and W-13.

Small peptides based on the CaMKII protein sequence are also of use in accordance with the present invention. Such small peptides include, e.g., CaMKII 290-309 (i.e., LKKFNARRKLKGAILTTMLA; SEQ ID NO:1), [Ala286]CaMKII Inhibitor 281-301 (i.e., MHRQEAVDCLKKFNARRKLKG; SEQ ID NO:2), and CaMKII. Inhibitor 281-309 (i.e., MHRQETVDCLKKFNARRKLKGAILTTMLA; SEQ ID NO:3). Similar longer, shorter, and neighboring protein sequences are also contemplated.

CaMKII inhibitors can also be based on the use of nucleic acid-based techniques to block the expression of CaMKII, and, therefore, to perturb the activity of CaMKII. Polynucleotide gene products are useful in this endeavor include antisense polynucleotides, ribozymes, RNAi, and triple helix polynucleotides that modulate the expression of CaMKII. Antisense polynucleotides and ribozymes are well-known to those of skill in the art. See, e.g., Crooke and B. Lebleu, eds., “Antisense Research and Applications” (1993) CRC Press; and “Antisense RNA and DNA” (1988) D. A. Melton, Ed., Cold Spring Harbor Laboratory Cold Spring Harbor, N.Y. Antisense RNA and DNA molecules act to directly block the translation of mRNA by binding to targeted mRNA and preventing protein translation. An example of an antisense polynucleotide is an oligodeoxyribonucleotide derived from the translation initiation site, e.g., between −10 and +10 regions of the relevant nucleotide sequence.

Although antisense sequences may be directed against the full-length genomic or cDNA of CaMKII, they also can be shorter fragments or oligonucleotides, e.g., polynucleotides of 100 or less bases. Although shorter oligomers (8-20) are easier to prepare and are more permeable in vivo, other factors also are involved in determining the specificity of base pairing. For example, the binding affinity and sequence specificity of an oligonucleotide to its complementary target increases with increasing length. It is contemplated that oligonucleotides of 8, 9, 10, 11, 12, 13, 14, 15, 16, 17, 18, 19, 20, 25, 30, 35, 40, 45, 50, or more base pairs will be used.

Ribozymes are enzymatic RNA molecules capable of catalyzing the specific cleavage of RNA. The mechanism of ribozyme action involves sequence specific interaction of the ribozyme molecule to complementary target RNA, followed by an endonucleolytic cleavage. Within the scope of the invention are engineered hammerhead or other motif ribozyme molecules that specifically and efficiently catalyze endonucleolytic cleavage of RNA sequences encoding protein complex components.

Specific ribozyme cleavage sites within any potential RNA target are initially identified by scanning the target molecule for ribozyme cleavage sites which include the following sequences, GUA, GUU, and GUC. Once identified, short RNA sequences of between 15 and 20 ribonucleotides corresponding to the region of the target gene containing the cleavage site can be evaluated for predicted structural features, such as secondary structure, that may render the oligonucleotide sequence unsuitable. The suitability of candidate targets also can be evaluated by testing their accessibility to hybridization with complementary oligonucleotides, using ribonuclease protection assays. See, WO 93/2356; and U.S. Pat. No. 5,093,246.

Nucleic acid molecules used in triple helix formation for the inhibition of transcription generally are single stranded and composed of deoxyribonucleotides. The base composition is designed to promote triple helix formation via Hoogsteen base pairing rules, which generally require sizeable stretches of either purines or pyrimidines to be present on one strand of a duplex. Nucleotide sequences can be pyrimidine-based, which results in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules can be selected that are purine-rich, for example, containing a stretch of G residues. These molecules form a triple helix with a DNA duplex that is rich in GC pairs, wherein the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Another technique that is of note for reducing the expression of a gene is RNA interference (RNAi). RNAi allows for the selective knockout of a target gene in a highly effective and specific manner. This technique involves introducing into a cell double-stranded RNA (dsRNA), having a sequence corresponding to a portion of the target gene. The dsRNA causes a rapid destruction of the target gene's mRNA. See, e.g., Hammond, et al. (2001) Nature Rev. Gen. 2:110-119; Sharp (2001) Genes Dev. 15:485-490. Methods and procedures for successful use of RNAi technology are well-known in the art, and have been described in, for example, Waterhouse, et al. (1998) Proc. Natl. Acad. Sci. USA 95(23):13959-13964. A short interfering RNA (siRNA) denotes a small interfering RNA that has a sequence complementary to a sequence within the target gene. Typically, siRNAs are about 20 to 23 nucleotides in length. The target sequence that binds the siRNA can be selected experimentally or empirically. For example, empirical observations have indicated that shRNA oligonucleotides targeting the transcriptional start site of the target gene (Hannon (2002) Nature 418:244-51) or targeting the 3′ untranslated region of the mRNA (He and Hannon (2004) Nature 5:522-531) are more effective at blocking gene expression. Further, siRNA target sites in a gene of interest are selected by identifying an AA dinucleotide sequence, typically in the coding region, and not near the start codon (within 75 bases) as these may be richer in regulatory protein binding sites which can interfere with binding of the siRNA (see, e.g., Elbashir, et al. (2001) Nature 411: 494-498). The subsequent 19-27 nucleotides 3′ of the AA dinucleotide can be included in the target site and generally have a G/C content of 30-50%.

RNAi can be performed, for example, using chemically-synthesized RNA. Alternatively, suitable expression vectors can be used to transcribe such RNA either in vitro or in vivo. In vitro transcription of sense and antisense strands (encoded by sequences present on the same vector or on separate vectors) can be effected using for example T7 RNA polymerase, in which case the vector can contain a suitable coding sequence operably-linked to a T7 promoter. The in vitro-transcribed RNA can, in certain embodiments, be processed (e.g., using E. coli RNase III) in vitro to a size conducive to RNAi. The sense and antisense transcripts are combined to form an RNA duplex which is introduced into a target cell of interest. Other vectors, e.g., lentiviral vectors, can be used that express small hairpin RNAs (shRNAs) which are processed into siRNA-like molecules. Various vector-based methods are described in, for example, Brummelkamp, et al. (2002) Science 296(5567):550-3; Lee, et al. (2002) Nat. Biotechnol. 20(5):500-5; Miyagashi and Taira (2002) Nat. Biotechnol. 20(5):497-500; Paddison, et al. (2002) Proc. Natl. Acad. Sci. USA 99(3):1443-8; Paul, et al. (2002); and Sui, et al. (2002) Proc. Natl. Acad. Sci. USA 99(8):5515-20. Various methods for introducing such vectors into cells, either in vitro or in vivo (e.g., gene therapy) are known in the art.

Kits for production of dsRNA for use in RNAi are available commercially, e.g., from New England Biolabs, Inc. and Ambion Inc. (Austin, Tex., USA). Methods of transfection of dsRNA or plasmids engineered to make dsRNA are routine in the art. Indeed, given the efficacy of an VEGFR1 siRNA developed by Sirna Therapeutics (San Francisco, Calif.; Singerman (2009) Retina 29(6 Suppl):S49-50) for the treatment of age-related macular degeneration, one of skill in the art can appreciate dosing of such molecules for achieving the desired therapeutic result with no systemic or local adverse events.

An siRNA molecule of the invention can encompass any siRNA that can modulate the selective degradation of CaMKII mRNA. In accordance with the present invention, particular embodiments embrace an siRNA molecule which selectively targets the alpha isoform of CaMKII. In specific embodiments, the siRNA molecule is set forth in SEQ ID NOs:6 and 7.

Antisense RNA and DNA molecules, ribozymes, RNAi and triple helix molecules can be prepared by any method known in the art for the synthesis of DNA and RNA molecules. These include techniques for chemically synthesizing-oligodeoxy-ribonucleotides well-known in the art including, but not limited to, solid phase phosphoramidite chemical synthesis. Alternatively, RNA molecules can be generated by in vitro and in vivo transcription of DNA sequences encoding the RNA molecule. Such DNA sequences may be incorporated into a wide variety of vectors which incorporate suitable RNA polymerase promoters such as the T7 or SP6 polymerase promoters. Alternatively, cDNA constructs that synthesize RNA constitutively or inducibly, depending on the promoter used, can be introduced stably or transiently into cells.

Particular embodiments of the present invention embrace the use of KN93, KN62, CaMKII Inhibitor 281-309, phenothiazines, siRNA and mixtures thereof. In specific embodiments, the inhibitor is specific for the alpha isoform of CaMKII.

Other suitable compounds having CaMKII inhibitory activity can be readily identified based upon the ability to alter or modulate protein levels or activities of components of the CaMKII pathway or activities regulated by the CaMKII pathway. In such a screening assay, a test compound is contacted with one or more components of the CaMKII pathway and the ability of said compound to decrease or reduce the level or activity of a component is measured. The level or activity of a component in the presence of the test compound can be compared to the level or activity of a component in the absence of the test compound to determine the effectiveness of the compound. Components of the CaMKII pathway embraced by the present invention include, calmodulin (CaM), Ca2+/CaM-dependent protein kinase II (CaMKII), plasma membrane calcium pumps (e.g., calcium-dependent K+/Ca+ pumps), calcium-dependent ATPase, calcium-dependent adenylate cyclase, type I phosphodiesterase or the protein phosphatase calcineurin. It is contemplated that the level or activity of the one or more components is measured using a cell-based or an in vitro assay. In a cell-based assay, a test cell is contacted with a test compound and the cell is subsequently monitored for the appearance of a phenotype associated with a decrease in calmodulin signaling (e.g., phosphorylation of a downstream protein). Alternatively, the level or activity of a CaMKII pathway component is determined using standard techniques such as immunoassays or enzyme activity assays specific to the protein being analyzed. More desirably, an in vitro assay is used for measuring the activity of the one or more components of the CaMKII pathway. In vitro assays for CaMKII pathway components are well-known in the art and many are commercially available, e.g., SignaTECT® CaMKII Assay System, Promega®, Madison, Wis.; Lehel, et al. ((1997) Anal. Biochem. 244(2):340-6); and BIOMOL GREEN™ Calcineurin Assay Kit. To further evaluate the efficacy of a compound identified using such screening assays, one can utilize the model systems disclosed herein to evaluate the adsorption, distribution, metabolism and excretion of a compound as well as its potential toxicity in acute, sub-chronic and chronic studies.

While some embodiments embrace the use of one or more CaMKII inhibitors in the treatment of pain, other embodiments embrace the use of one or more CaMKII inhibitors used in combination with an opiate analgesic in the treatment of pain. Accordingly, the dose of opiate analgesic can be reduced, while providing an analgesic effect equivalent to administering a higher dose of opiate alone. The reduced dose of opiate also reduces adverse side effects associated with opiate administration, and can significantly reduce the addiction potential of opiate in susceptible individuals.

Morphine is an important drug for the treatment of moderate to severe pain. Morphine primarily is used to treat severe pain associated with trauma, myocardial infarction, and cancer. Although, morphine is one of the most effective painkillers, effective pain management requires that adequate analgesia be achieved without excessive adverse side effects. Many patients treated with morphine are not successfully treated because of excessive adverse side effects and/or inadequate analgesia. For example, the use of morphine in the treatment of chronic pain is limited because of inadequate analgesia. Research efforts have been directed to the development of opioid analgesics, however the problem of tolerance to, and dependence on, these agonists persists (Williams, et al. (2001) Physiol. Rev. 81:299-343). For example, the chronic administration of morphine results in the development of physical dependence, as evidenced by the appearance of distressing physical symptoms induced by abrupt termination of morphine treatment. The signs and symptoms simulate a severe cold, and usually include nasal discharge, lacrimation, chills, goose pimples, muscular aches, enhanced motor reflexes, profound body water loss attributed to hyperthermia, hyperventilation, emesis, and diarrhea. It is well-known that various types of opioid receptors are involved in the development of the psychological and physical dependence on opioids.

Accordingly, to increase the effectiveness of opiate analgesics, the present invention embraces consecutive or simultaneous administration of a CaMKII inhibitor with an opiate analgesic. Desirably, the opiate analgesic is administered with a CaMKII inhibitor in a weight ratio of analgesic-to-inhibitor of about 0.01:1 to about 100:1, preferably about 0.02:1 to about 50:1, and most preferably about 0.1:1 to about 10:1. This ratio depends upon the type and identity of opiate analgesic and CaMKII inhibitor being used and the origin and severity of the pain being treated. This ratio can be readily determined by a person skilled in the art to achieve the desired reduction in pain. Opiate analgesics of use in accordance with the present invention include one or more opium alkaloids or semisynthetic opiate analgesics. Specific opiate analgesics include, but are not limited to, opium; opium alkaloids, such as morphine, morphine sulfate, codeine, codeine phosphate, codeine sulfate, diacetylmorphine, morphine hydrochloride, morphine tartrate, and diacetylmorphine hydrochloride; and semisynthetic opiate analgesics, such as dextromethorphan hydrobromide, hydrocodone bitartrate, hydromorphone, hydromorphone hydrochloride, levorphanol tartrate, oxymorphone hydrochloride, and oxycodone hydrochloride. Other opioids include, but are not limited to, fentanyl, meperidine, methodone, and propoxyphene.

In accordance with another important feature of the present invention, it has been discovered that chronic actions of morphine and related opioids (e.g., tolerance and dependence, as well as opioid-induced hyperalgesia (OIH)), but not the acute action of morphine and related opioids (e.g., analgesia), can be modulated by CaMKII inhibitors. This approach is particularly useful for the management of symptoms of morphine withdrawal.

The present invention also is directed to the administration of a CaMKII inhibitor to an individual undergoing an opiate analgesic therapy to prevent, reduce or reverse opiate analgesic tolerance or OIH in the individual. The administration of a CaMKII inhibitor allows the dose of an opiate analgesic to remain constant, or to be reduced, while maintaining the desired pain-reducing effect. By reducing or reversing tolerance or OIH to an opiate analgesic, the occurrence of adverse side effects can be reduced, and the possibility of opiate analgesic dependence is reduced. Moreover, in so far as OIH is defined as a lowered pain threshold caused by opioid exposure, the prevention, reduction or reversal of OIH would render a patent less sensitive to pain as compared to a subject not receiving a CaMKII inhibitor.

The present invention, therefore, provides compositions and methods of preventing, reducing or reversing tolerance or OIH to opiate analgesics, thus potentiating the analgesic properties of an opiate analgesic. The phrase “reducing or reversing opiate analgesic tolerance” is defined as the ability of a compound to reduce the dosage of an opiate analgesic administered to an individual to maintain a level of pain control previously achieved using a greater dosage of opiate analgesic. The present invention also provides pharmaceutical compositions comprising an opiate analgesic and a CaMKII inhibitor. Further provided are articles of manufacture containing an opiate analgesic and a CaMKII inhibitor, packaged separately or together, and an insert having instructions for using the active agents.

The methods described herein benefit from the use of an opiate analgesic and a CaMKII inhibitor in the treatment and management of pain. The analgesic and CaMKII inhibitor can be administered simultaneously or sequentially to achieve the desired effect of pain treatment or reduction or reversal of opiate analgesic tolerance.

While the compounds disclosed herein, as well as those identified in screening methods, are useful in the treatment of pain including both acute and chronic pain, particular embodiments of the present invention provide for the treatment of chronic pain. The distinction between acute and chronic pain is not based on its duration of sensation, but rather the nature of the pain itself. In general, acute pain is distinguished by having a specific cause and purpose, and generally produces no persistent psychological reaction. The primary distinction is that acute pain serves to protect one after an injury, whereas chronic pain does not serve this or any other purpose. Acute pain is the symptom of pain, chronic pain is the disease of pain. Chronic pain in accordance with the present invention includes cancer pain, post-traumatic pain, post-operative pain, neuropathic pain, inflammatory pain or pain associated with a myocardial infarction. Neuropathic pain is chronic pain resulting from injury to the nervous system. The injury can be to the central nervous system (brain or spinal cord) or the peripheral nervous system. Neuropathic pain can occur after trauma or as a result of diseases such as multiple sclerosis or stroke. Accordingly, the compounds of the present invention are particularly useful for treating neuropathies, polyneuropathies (e.g., diabetes, headache, and trauma), neuralgias (e.g., post-zosterian neuralgia, postherpetic neuralgia, trigeminal neuralgia, algodystrophy, and HIV-related pain); musculo-skeletal pain such as osteo-traumatic pain, arthritis, osteoarthritis, spondylarthritis as well as phantom limb pain, back pain, vertebral pain, chipped disc surgery failure, post-surgery pain; cancer-related pain; vascular pain such as pain resulting from Raynaud's syndrome, Horton's disease, arteritis, and varicose ulcers; as well as pain associated with multiple sclerosis, Crohn's Disease, and endometriosis.

Prevention or treatment of pain is accomplished by delivering an effective amount of a compound disclosed herein to a subject in need of treatment, i.e., a subject about to experience pain (e.g., a surgical patient) or a subject already experiencing inflammatory and neuropathic pain. In most cases this will be a human being, but treatment of livestock, zoological animals and companion animals, e.g., dogs, cats and horses, is expressly covered. The selection of the dosage or effective amount of a compound of the invention is that which has the desired outcome of preventing, ameliorating or reducing at least one symptom associated with pain. As such, compounds of the present invention find application in both medical therapeutic and/or prophylactic administration, as appropriate. Effectiveness of a compound of the invention may reduce behavioral hypersensitivity of inflammatory pain or neuropathic pain. Behavioral hypersensitivity of pain can include sensations that are sharp, aching, throbbing, gnawing, deep, squeezing, or colicky in nature and can be measured by, for example, exposure to thermal hyperalgesia or mechanical allodynia.

For administration to a subject, a compound of the invention is generally formulated in a pharmaceutical composition containing the active compound in admixture with a suitable carrier. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier, composition or vehicle, such as a liquid or solid filler, diluent, excipient, or solvent encapsulating material, is involved in carrying or transporting the subject compound from one organ, or portion of the body, to another organ, or portion of the body. Each carrier must be acceptable in the sense of being compatible with the other ingredients of the formulation and not injurious to the subject being treated.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The compositions of the present invention can be administered parenterally (for example, by intravenous, intraperitoneal, subcutaneous or intramuscular injection), topically (including buccal and sublingual), orally, intranasally, intravaginally, intrathecally or rectally, with oral and intrathecal administration encompassing particular embodiments of this invention.

The selected dose to be administered will depend upon a variety of factors including the activity of the particular compound of the present invention employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well-known in the medical arts. A physician or veterinarian having ordinary skill in the art can readily determine and prescribe the effective amount of the pharmaceutical composition required based upon the teachings herein and standard medical practices. For example, the physician or veterinarian could start doses of the compounds of the invention employed in the pharmaceutical composition at levels lower than that required in order to achieve the desired therapeutic effect and gradually increase the dosage until the desired effect is achieved. This is considered to be within the skill of the artisan and one can review the existing literature on a specific compound to determine optimal dosing.

The finding that spinal CaMKII is an essential mediator of pain and opioid tolerance is determined by the following experiments. In addition, the studies correlate the inhibition of CaMKII with opioid tolerance. The results of the present studies illustrate the role of spinal CaMKII in opioid tolerance, which led to the present novel pain therapy.

The invention is described in greater detail by the following non-limiting examples.

### Example 1

### Materials and Methods

Materials.

Complete Freund's adjuvant (CFA, 1 mg/ml Mycobacterium tuberculosis (H 37RA, ATCC 25177, Heat killed and dried) and Trifluoperazine were purchased from Sigma (St. Louis, Mo.). KN93 [2-[N-(2-hydroxyethyl)]-N-(4-methoxybenzenesulfonyl)]amino-N-(4-chlorocinnamyl)-N-methylbenzylamine)] and KN92 [2-[N-(4-methoxybenzenesulfonyl)] amino-N-(4-chlorocinnamyl)-N-methylbenzylamine] were from Calbiochem (Gibbstown, N.J.). Morphine sulfate, morphine and placebo pellets were obtained from the National Institute on Drug Abuse (Rockville, Md.). Protease inhibitor Cocktail Tablets were from Roche Diagnostics (Mannheim, Germany). Haloperidol, naloxone and all the other chemical reagents were from Sigma (St. Louis, Mo.).

Cell Lines.

Human neuroblastoma SH-SY5Y cells were maintained as a monolayer culture in Dulbecco's Modified Eagle Medium (DMEM) supplemented with 10% fetal calf serum, 100 μg/ml streptomycin and 100 units/ml penicillin in 5% carbon dioxide with the incubator maintained at 37° C. Cells were plated into flasks a week before experiments. Treatments were terminated at the designated times by replacing the medium with ice-cold phosphate-buffered saline (PBS) on ice and subsequently rinsed with PBS three times. Cells were then used in assays.

Animals.

Male ICR mice (20-25 grams; Harlan Laboratories, Indianapolis, Ind.) were kept in a vivarium, with a 12 hour or 14/10 hour alternating light-dark cycle with food and water available ad libitum before experimental procedures. All experiments were performed during the light cycle. Mice were randomly divided into experimental groups according to a computer generated randomization list (n is as indicated in each group). Each animal was used in one experiment only. All study personnel were blinded to treatment assignment for the duration of the study. All procedures were performed in accordance with the policies and recommendations of the International Association for the Study of Pain and the National Institutes of Health guidelines for the handling and use of laboratory animals.

Male Sprague-Dawley rats (250-350 grams) were used in all other experiments.

CaMKIIα^{T286A} mutant mice (Giese, et al. (1998) Science 279:870-873) were back-crossed with C57Bl6/J mice for 10 generations. Heterozygous breeding was used to generate male homozygous mutant mice and littermate wild-type control mice for this study. Both genotypes were viable and showed normal growth and reproduction. Genotyping of litters was performed by PCR using a set of primers (5′-CTG TAC CAG CAG ATC AAA GC-3′, SEQ ID NO:4 and 5′-ATC ACT AGC ACC ATG TGG TC-3′, SEQ ID NO:5). The PCR products for wild-type and mutant alleles were 200 and 290 bp, respectively. Each experimental mouse was genotyped twice using DNA from two separate extractions from tail tissue samples. The investigators who performed the biochemical and behavioral tests were blind to mouse genotypes.

CFA-Induced Inflammatory Pain.

Unilateral inflammation was induced by injecting 20 μl CFA into the plantar surface of the mouse left hindpaw. Mice were tested for thermal hyperalgesia and tactile allodynia before and 24 hours and 72 hours after CFA injection (before and 30 minutes after intrathecal or intraperitoneal drug administration).

Spinal Nerve Ligation (SNL).

SNL was performed as using established methods (Kim & Chung (1992) Pain 50:355-63; Wang, et al. (2001) J. Neurosci. 21:1779-86). Groups of eight mice had the L5 and L6 spinal nerve tightly ligated distal to the dorsal root ganglion but before the fibers joined the sciatic nerve; sham operation consisted of the same procedures but without the ligation. Mice were tested for thermal hyperalgesia and tactile allodynia before and after SNL operation (30 minutes after intrathecal or intraperitoneal drug administration).

Drug Administration.

Intrathecal injection (i.t.) was given in a volume of 5 μl by percutaneous puncture through an intervertebral space at the level of the 5th or 6th lumbar vertebra of mice, according to a previously reported procedure (Hylden & Wilcox (1980) Eur. J. Pharmacol. 67:3134-316; Wang, et al. (2001) supra) using a Hamilton microsyringe with a 30-gauge needle. Success of i.t. injection was verified by a lateral tail flick.

For KN93 studies, pre-treatment group was given KN93 (30 nmol i.t.) 1 hour before CFA injection followed by two additional injections at the same dose at 24 hours and 72 hours after CFA injection; post-treatment group was only administrated KN93 (30 nmol or 45 nmol, i.t.) or KN92 (45 nmol i.t.) at 24 hours and 72 hours after CFA injection. For SNL model, KN93 or KN92 (45 nmol, i.t.) was given on day 5, 30 minutes before behavior testing.

Trifluoperazine (0.1 mg/kg, 0.25 mg/kg, 0.5 mg/kg) was administrated by intraperitoneal injection (i.p.) 1 day after CFA injection or 5 days after SNL. Equal volume of saline was used as control for drugs.

For i.t. drug administration in rats, the method of Sakura, et al. (1996) Anesthesiology 85:1184-1189) was employed. While under isoflurane anesthesia, an 8 cm length of PE10 tubing (32 gauge) was inserted through an incision made in the atlantooccipital membrane to the level of the lumbar enlargement. The catheter then was secured to the musculature at the site of incision, which then was closed. The rats were allowed 5 to 7 days to recover before experiments began. Rats exhibiting signs of motor deficiency were euthanized. Intrathecally administered substances were dissolved in saline and administered in a volume of 5 μl through a tubing with calibrated length connecting the i.t. catheter with the injection syringe. Progress of the injection was monitored carefully by observing the movement of a small air bubble (1 μl in volume) through the tubing. The catheter was cleared by flushing with 9 μl saline. In all cases, a dye was injected into the cannula at the termination of the experiment to ensure correct i.t. placement.

To induce opioid tolerance, rats were subcutaneously implanted with two morphine pellets or placebo pellets (75 mg morphine base in each pellet, wherein placebo contained no morphine; National Institute on Drug Abuse, Rockville, Md.) according to well-established protocols. Briefly, while under isoflurane anesthesia, a 1.5-cm incision was made on the back of the animal. Morphine and placebo pellets were implanted into the subcutaneous space and the incision was closed with wound clips. These pellets were left for less than 7 days unless otherwise indicated.

For haloperidol reversion tests, haloperidol (0.06, 0.20, 0.60 mg/kg respectively i.p.) was given 0.5 hours before the test dose of morphine (10 mg/kg s.c.). For prevention test, same dose of haloperidol was given 0.5 hours before the induce dose of morphine (100 mg/kg s.c.). Antinociception effects were measured 0.5 hours after the test dose of morphine. Morphine dependence was evaluated by recording the naloxone (10 mg/kg i.p.)-induced jumping.

To inhibit CaMKIIα, CaMKIIα was targeted by small interfering RNA (siRNA). Four days after morphine pellet implantation, mice were treated with CaMKIIα siRNA (5′-CAC CAC CAU UGA GGA CGA AdTdT-3′, SEQ ID NO:6 and 3′-dTdTG UGG UGG UAA CUC CUG CUU-5′, SEQ ID NO:7) (Zayzafoon, et al. (2005) J. Biol. Chem. 280:7049-7059) or STEALTH™ RNAi negative control (Invitrogen, Carlsbad, Calif.) (2 μg, twice/day for 3 consecutive days, i.t.). These oligos were mixed with the transfection reagent I-FECT™ (Neuromics, Minneapolis, Minn.), in a ratio of 1:5 (W:V) (Luo, et al. (2005) Mol. Pain 1:29). Mechanical and thermal sensitivity tests were performed daily.

Thermal Hyperalgesia.

The paw withdrawal latencies to heat stimuli were measured using a plantar test (model 7372; UGO BASILE, VA, Italy) according to known methods (Hargreaves, et al. (1988) Pain 32:77-88; Wang, et al. (2001) supra). Mice were placed under a clear plastic cage on a glass floor. After a 30-minute period of habituation, paw withdrawal latencies to radiant heat stimulation were measured. The radiant heat source was focused on the middle portion of the plantar surface of the left hindpaw, which was automatically ceased when a paw withdrawal occurred. A cut-off time of 20 seconds was applied in order to prevent skin damage.

Mechanical Allodynia.

Mechanical allodynia or mechanical sensitivity was measured using calibrated von Frey filaments (Stoelting, Wood Dale, Ill.). Briefly mice were placed into individual plexiglas container with a wire mesh floor and allowed to acclimate for 30 minutes before testing. Each von Frey filament was applied perpendicularly to the mid-plantar surface for 5 seconds or until a withdrawal response occurred. The up-down paradigm was used to determine 50% probability paw withdraw threshold (Chaplan, et al. (1994) J. Neurosci. Methods 53:55-63; Dixon (1980) Annu. Rev. Pharmacol. Toxicol. 20:441-62; Wang, et al. (2001) supra).

Antinociception Tests.

Tail flick test was used for basal nociception and morphine induced antinociception as described previously (Wang, et al. (2001) supra). In brief, ⅓ of distal mice tails were immersed in to a water bath maintained at 52° and latency of the tail-flick response was recorded. Morphine-induced (s.c.) antinociception effect was evaluated at drug peak response time after the morphine sulfate injection and represented as percentage of maximal possible effect (% MPE). % MPE=100%*(postdrug latency−predrug latency)/(cut-off−predrug latency). A 12 second cut-off time was used to prevent tissue damage. Thirty minute drug peak response time was determined and was not altered by opioid tolerance.

Opioid Tolerance and Dependence.

To induce an acute model of opioid tolerance and dependence, ICR mice (20˜25 grams) were administrated a large dose of morphine sulfate (100 mg/kg s.c.). The maximal morphine tolerance and dependence was developed over 2-6 hours as reported (Bilsky, et al. (1996) supra). The same volume of saline was given to control mice. A test dose of morphine (10 mg/kg s.c.) was given 4.5 hours later and opioid tolerance was evaluated by measuring the antinociception effect 0.5 hours after the test dose of morphine. A significant reduction of antinociception indicated the presence of morphine tolerance. Morphine dependence was evaluated by naloxone-induced withdraw test. Mice were given naloxon (10 mg/kg i.p.) right after the antinociception effect was measured and immediately kept inside glass beaker. Vertical jumps were recorded for 15 minutes.

Opioid-Induced Hyperalgesia (OIH) by Subcutaneous Administration.

To induce OIH, mice were treated subcutaneously according to an established schedule (Liang, et al. (2006) Anesthesiology 104:1054-1062). Mice received mg/kg morphine sulfate (twice per day, s.c.) for 3 consecutive days and two more injections of 40 mg/kg morphine sulfate on day 4. Control mice received equal volume and number of saline injections (s.c.). Mechanical and thermal sensitivities were tested before and after morphine treatment.

OIH Induced by Morphine Pellet Implantation.

Separate groups of 5 male ICR mice were implanted subcutaneously with morphine pellets or placebo pellets (1 pellet/mouse, each morphine pellet contains 75 mg morphine base; a placebo pellet is contains no morphine). This treatment not only induces opioid dependence and tolerance (Tang, et al. (2006) J. Pharmacol. Exp. Ther. 317:901-909), but also OIH (Li, et al. (2001) Brain Res. Mol. Brain Res. 86:56-62). In order to determine the presence of OIH, mechanical and thermal sensitivities were tested before and after morphine treatment as described below.

siRNA Administration

Western Blot Analysis.

Brain and lumbar sections of spinal cord from mice were quickly dissected and frozen on dry ice. Tissues from three mice of the same experimental group were pooled and homogenized using a glass homogenizer in 200 μL RIPA buffer (1% NP-40, 0.5% sodium deoxycholate, 0.1% sodium dodecyl sulfate (SDS), 5 mM EDTA in PBS, pH 7.4) in the presence of protease inhibitors (0.05 mg/ml bestatin, 0.05 mg/ml leupeptin, 0.05 mg/ml pepstatin, and 0.1 mg/ml phenylmethylsulfonylfluoride). The homogenates were incubated on rotator at 4° C. for 1-2 hours, and the soluble fraction was separated by centrifugation (45,000×g, 45-60 minutes). Protein content in the supernatant was determined by a modified Bradford method (Pierce Biotechnology, Rockford, Ill.). Samples (15-60 μg of protein) were separated by 10% SDS-PAGE and electrotransferred onto PVDF membrane. The membrane was preblocked in 5% non-fat milk in 20 mM Tris-buffer saline (pH 7.6) with 0.1% Tween and probed with a rabbit anti-(T286)pCaMKIIα antibody (1:1000; Promega, Madison, Wis.) or a mouse anti-CaMKIIα antibody (1:1,000, Santa Cruz Biotechnology) at room temperature for 3 hours. The membrane was then washed and incubated with a donkey anti-rabbit IgG-HRP conjugate secondary antibody (1:1000; Amersham, Piscataway, N.J.) (for pCaMKIIα) or anti-mouse (for CaMKIIα) secondary antibody (1:1,000, GE Healthcare, Piscataway, N.J.), washed and developed using an enhanced chemiluminescence detection system (ECL; Amersham, Piscataway, N.J.). The membrane was then stripped and reprobed with a mouse anti-β-actin antibody (1:10,000; Sigma) followed by another incubation with anti-mouse HRP-conjugated secondary antibody (1:20,000; Amersham) and developed as above. ECL-signals were captured by a ChemiDoc imaging system and analyzed using Quantity One program (BIO-RAD, Hercules, Calif.). To control protein loading, β-actin level was measured in the same immunoblot using monoclonal anti-β-actin antibody ( 1/10,000; Sigma Aldrich, St. Louis, Mo.) for 1 hour followed by horseradish-peroxidase-linked anti-mouse secondary antibody IgG ( 1/10,000; Amersham Biosciences, Piscataway, N.J.) for 1 hour, then developed as above. The band density was measured with Quantity One Program (BIO-RAD Hercules, Calif.). CaMKII immunoreactivity was expressed as the ratio of the optical densities of pCaMKII or CaMKIIα to those of β-actin.

Western blot analysis of samples from rats is as above. Sample preparation involved sacrificing the rats by carbon dioxide inhalation and decapitated. The spinal column was cut through at the S1/S2 level. A 16-gauge needle was inserted in the sacral vertebral canal, attached to a syringe containing ice-cold saline, and the spinal cord was ejected through the cervical opening. The spinal cord was placed on ice in a glass Petri dish and rapidly dissected using a dissecting microscope. For consistency, the lumbar enlargement corresponding to the L1 to L6 spinal segments was excised and used for all assays. Tissue samples were frozen immediately in liquid nitrogen and stored at −80° C. until analyzed.

Rotarod Test.

To exclude the possibility that KN93 may cause locomotor impairment, a rotarod test was performed (Prestori, et al. (2008) J. Neurosci. 28:7091-7103). Mice were trained to remain on a fixed speed (4 rpm) rotarod for 60 seconds (model series 8; IITC Life Science, Woodland Hills, Calif.). On the following day, mice were retrained and those that failed to stay on the rotarod for 60 seconds were not used in further studies. Baseline was tested 30 minutes later by placing the mice on an accelerating rotarod (4-40 rpm over 300 seconds). The latency to fall off the rotarod was recorded. Mice were then treated (i.t.) with either KN93 (45 nmol), KN92 (45 nmol) or saline and retested 0.5, 1, 2, 4, and 8 hours later. The cut-off was set at 300 seconds.

Immunohistochemistry.

Immunohistochemical staining of spinal CaMKII was carried out according to established methods (Wang, et al. (2001) J. Neurosci. 21:1779-1786). Mice were deeply anesthetized with ketamine (100 mg/kg) and xylazine (5 mg/kg, i.p.). The vascular system was perfused with 60 ml of 4° C. phosphate-buffered saline (PBS, pH 7.4) and followed by 50 ml of 2% paraformaldehyde solution. The lumbar spinal cords were removed, post-fixed overnight in 4% papraformaldehyde at 4° C., and cryoprotected in 30% sucrose for 24 hours at 4° C. Spinal cord sections were cut on a cryostat at 20 μm thickness and washed 2 times with cold PBS. Sections were pre-incubated with 0.3% H₂O₂ and blocked with 10% goat serum (Invitrogen, Carlsbad, Calif.) and 0.3% TRITON X-100 in PBS for minutes. Floated sections were incubated with the primary antibody for pCaMKIIαThr286 (1:1,000) overnight at room temperature, followed by another incubation with biotinylated goat anti-rabbit IgG secondary antibody (1:200, Vector Laboratories, Burlingame Calif.) at room temperature for 2 hours. Incubation in the absence of the first antibody or in the presence of pCaMKIIαThr286 blocking peptide (Santa Cruz) was used to control for antibody specificity. The sections were developed using ELITE VECTASTAIN ABC kit (Vector Laboratories). Diaminobenzidine (DAB)-stained sections were imaged by inverted microscopy (Olympus, Melville, N.Y.) and quantified using the MetaMorph Imaging Software (Universal Imagining, West Chester, Pa.). For each condition, 5 sections and 6 areas from each section were analyzed and averaged.

Statistical Analysis.

Data were presented as mean±S.E.M. Comparisons between groups were analyzed using a student t-test (two groups) or a two-way repeated measure ANOVA followed by post-hoc analyses using Dunnett's t-test (multiple groups). Statistical significance was established at 95% confidence limit.

### Example 2

### Prevention and Treatment of Pain with KN93

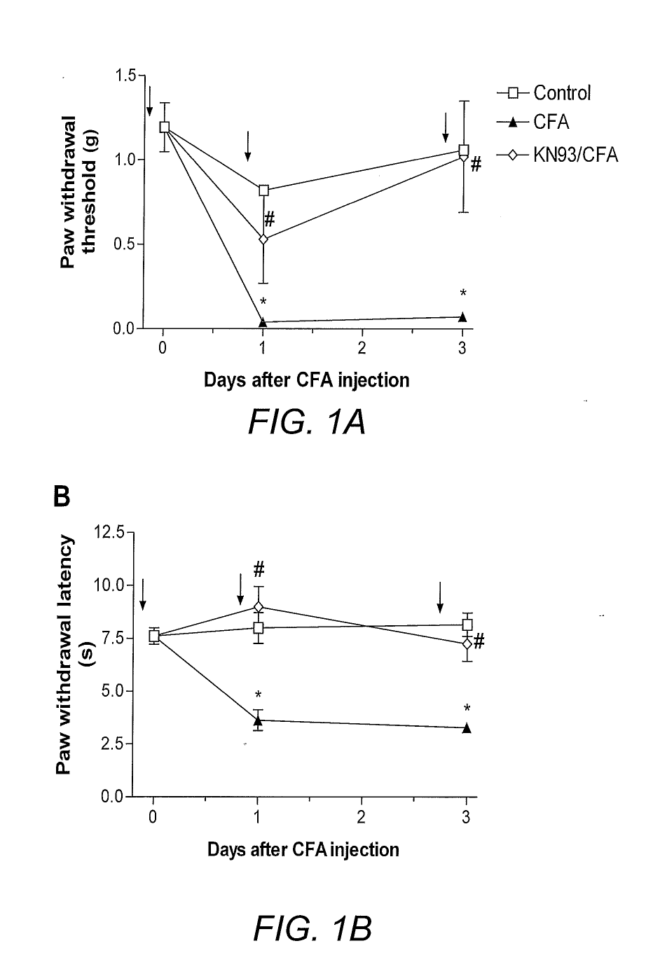

To illustrate the involvement of the CaMKII signaling pathway in pain, CaMKII inhibitors were employed in a rat model of inflammatory pain and mouse model of neuropathic pain. Intraplantar injection of complete Freund's adjuvant (CFA) into the rat hind paw to induce inflammation has been used as a reliable animal model of inflammatory pain (Iadarola, et al. (1988) Pain 35(3):313-26). For neuropathic pain, a spinal nerve ligation model (SNL; Kim and Chung (1992) Pain 50(3):355-63) is widely accepted. Thermal hyperalgesia and mechanical allodynia have been shown in many studies to develop after CFA injection (Iadarola, et al. (1988) supra) or SNL (Kim and Chung (1992) supra; Wang, et al. (2001) J. Neurosci. 21(5):1779-86). Consistent with the prior art, CFA-treated mice demonstrated significantly reduced withdrawal latency to radiant heat and decreased withdrawal threshold to von Frey filaments within 24 hours and 72 hours (FIGS. 1A and 1B). It was subsequently determined whether blocking the activation of CaMKII could block the development of pain behavior induced by CFA. Intrathecal administration of KN93 (30 nmol), a CaMKII inhibitor, 1 hour before CFA injection followed by two additional injections at the same dose on Day 1 and Day 3 significantly blocked the development of mechanical allodynia (FIG. 1A) and thermal hyperalgesia (FIG. 1B) during the entire experimental period (P<0.05 compared with CFA group, N=8).

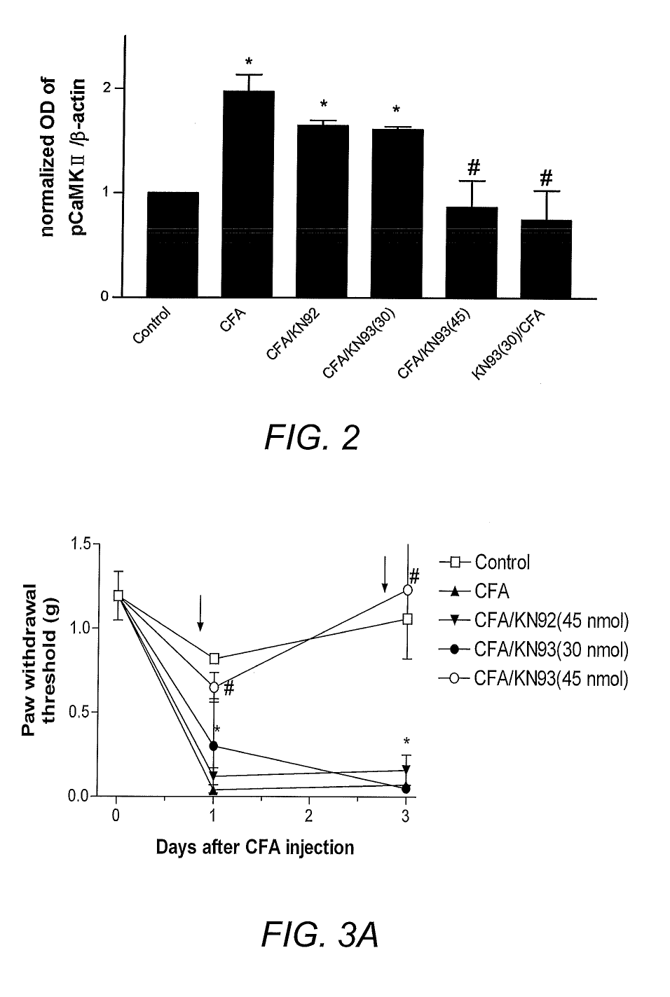

CaMKII activity (pCaMKII) after CFA injection was also examined. Western blot analysis indicated that pCaMKII was up-regulated by CFA on Days 1 and 3 (FIG. 2) (P<0.05, N=4), wherein pretreatment with KN93 (30 nmol, i.t.) prevented the CFA-induced increase of pCaMKII (FIG. 2) (P<0.05 compared with CFA group, N=4). In contrast, KN92, a kinase-inactive chemical analogue of KN93, did not alter the activity of CaMKII (FIG. 2).

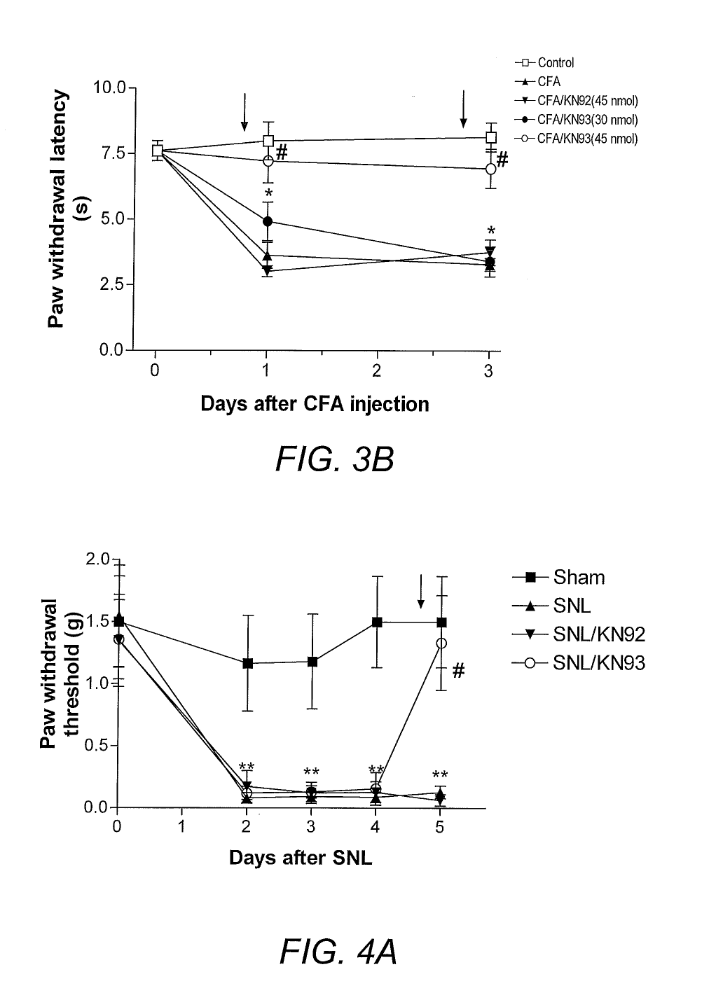

It was subsequently determined whether CaMKII inhibition could reverse established CFA-induced pain behavior. KN93 (45 nmol, i.t.) dose-dependently reversed mechanical allodynia (FIG. 3A) and thermal hyperalgesia (FIG. 3B) when tested on Days 1 and 3 after CFA injection (P<0.05, N=8, compared with CFA group). At a lower dose (30 nmol, i.t.), KN93 only produced a partial reversal. As depicted in FIG. 2, CFA-increased CaMKII activity was significantly attenuated by KN93 only at the higher dose (45 nmol) (P<0.05, n=4, compared with CFA group), but not by the lower dose (30 nmol) (P>0.05). Therefore, KN93 dose-dependently reversed mechanical allodynia and thermal hyperalgesia, which was consistent with the inhibitor's action on CaMKII. Treatment with KN92 (45 nmol, i.t.) did not affect CFA-induced allodynia (FIG. 3A) or hyperalgesia (FIG. 3B) (P>0.05, N=8, compared with CFA group). Likewise, KN92 had no effect on CaMKII activity (pCaMKII) (FIG. 2). At 45 nmol (i.t.), neither KN93 nor KN92 altered nociception baseline or caused gross behavior changes in naïve mice.

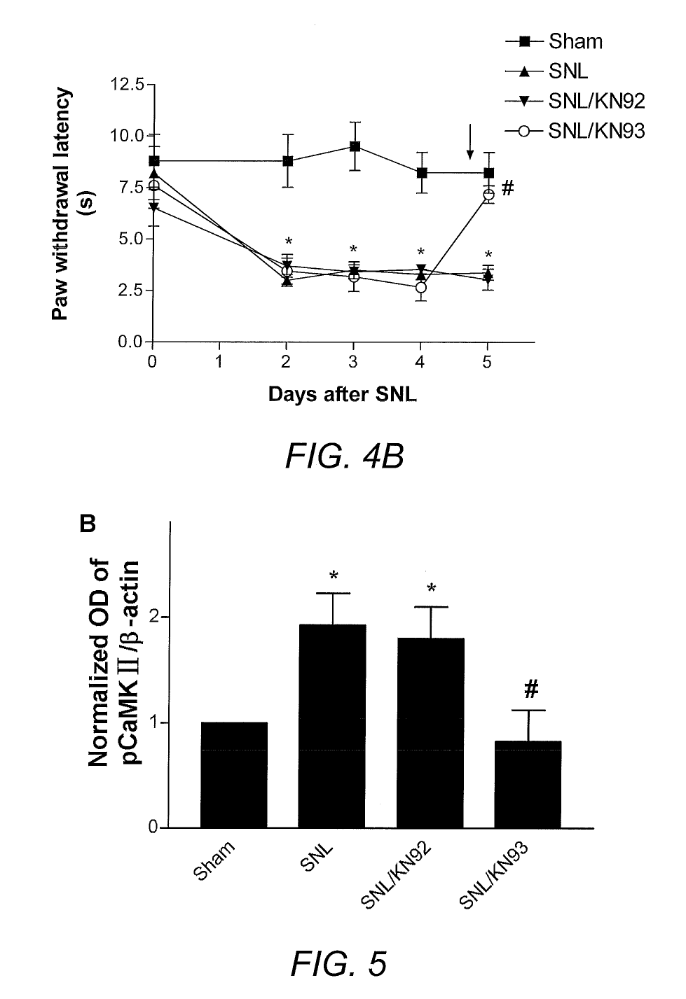

In a mouse model of neuropathic pain, KN93 (45 nmol, i.t.) fully reversed both mechanical allodynia (FIG. 4A) and thermal hyperalgesia (FIG. 4B) when administered 5 days after SNL (P<0.05, n=8, compared with SNL group). However, post-treatment with KN92 (45 nmol, i.t.) showed no effect on nociceptive threshold (P>0.05, n=8, compared with SNL group). With respect to CaMKII activity, western blot analysis indicated that CaMKII activity was up-regulated by the SNL treatment when compared with the Sham operation (P<0.05, N=3) (FIG. 5), wherein KN93 (45 nmol, i.t.) reversed the SNL-induced increase in pCaMKII activity on Day 5 (P<0.05, compared with the Sham group, N=4) (FIG. 5). KN92 did not affect the expression of pCaMKII (P>0.05 compared with the Sham group, N=4) (FIG. 5).

### Example 3

### Treatment of Pain with Anti-Psychotics which Inhibit CaMKII

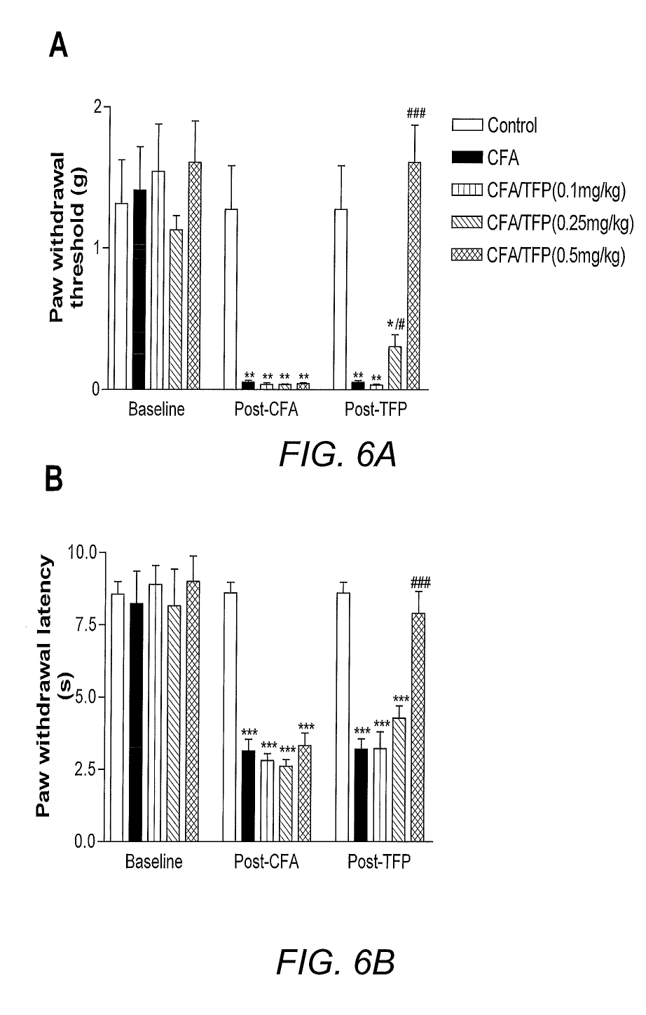

As indicated herein, trifluoperazine is a potent CaMKII inhibitor. As with KN93, trifluoperazine (0.5 mg/kg, i.p.) also completely reversed mechanical allodynia (FIG. 6A) and thermal hyperalgesia (FIG. 6B) induced by CFA (P<0.001 compared with the CFA group, N=8). At a lower dose (0.25 mg/kg, i.p.), trifluoperazine exhibited a partial effect in alleviating mechanical allodynia (P<0.05 compared with the CFA group, N=8). At an even lower dose (0.1 mg/kg, i.p.), this drug had no effect on CFA-induced hyperalgesia or allodynia (FIG. 6).

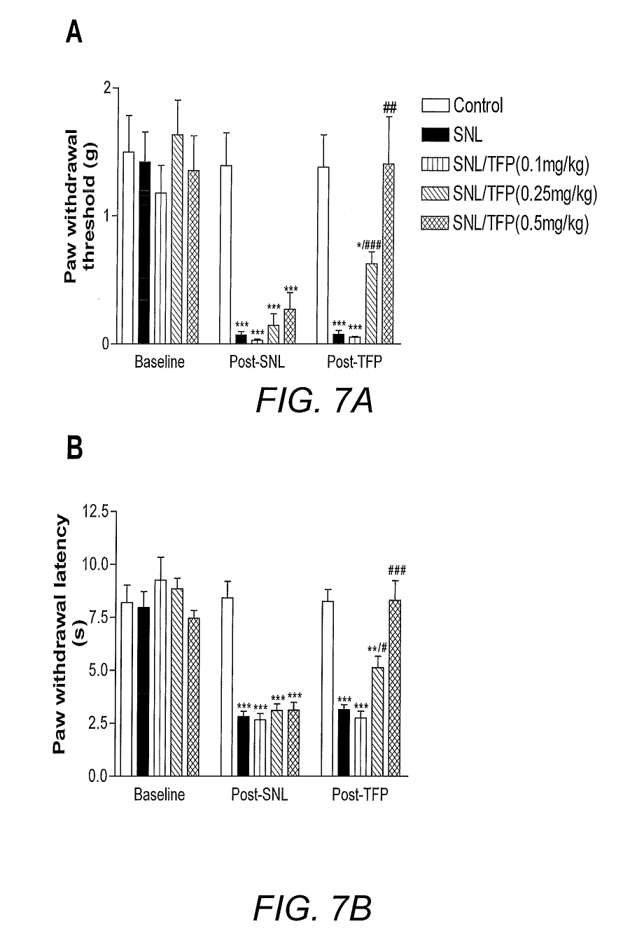

Similar to KN93, trifluoperazine (0.5 mg/kg, i.p., Day 5) also completely reversed established SNL-induced mechanical allodynia (P<0.01 compared with the SNL group, N=8) (FIG. 7A) and thermal hyperalgesia (P<0.001 compared with the SNL group, N=8) (FIG. 7B). The intermediate dose of trifluoperazine (0.25 mg/kg, i.p.) partially attenuated mechanical allodynia (P<0.001 compared with the SNL group, N=8) (FIG. 7A) and thermal hyperalgesia (P<0.05 compared with the SNL group, N=8) (FIG. 7B), whereas the drug was ineffective at the lowest dose tested (0.1 mg/kg, i.p.) (FIG. 7). Intraperitoneal injection of trifluoperazine (0.5 mg/kg) did not by itself alter nociceptive baseline (P>0.05 compared with the predrug baseline, N=12). These data indicate that trifluoperazine dose-dependently reverses CFA- and SNL-induced thermal hyperalgesia and tactile allodynia.

To demonstrate the general applicability of targeting the CaMKII signaling pathway for the treatment of pain, other similar anti-psychotic agents including chlorpromazine, chlorprothixene, clozapine, fluphenazine, haloperidol, perphenazine, pimozide, prochlorperazine, promazine, promethazine, and thioridazine were analyzed by injection and oral administration. As with KN93 and trifluoperazine, each of these agents inhibited CaMKII activity. Advantageously, most of these agents are atypical anti-psychotic drugs that can be taken orally and have been used in humans for decades.

### Example 4

### K93 Reverses Opioid Tolerance and Dependence

At the outset, tests were performed to determine whether a clinically used opioid receptor agonist, i.e., morphine, activates CaMKII in the human neuroblastoma SH-SY5Y cells. It was found that intracellular free calcium and calmodulin both increased after treatment with morphine in cultured cells, as did CaMKII activity.

After establishing a time course and dose-dependent activation of CaMKII (0.1 nM to 100 μM) with SH-SY5Y cells, tests were performed to confirm whether CaMKII regulation is effected by an opioid agonist in vivo, thereby correlating CaMKII expression and activity temporally to opioid tolerance. Subcutaneous implantation of morphine pellets (two 75 mg pellets) is well-established in the art for producing antinociceptive tolerance in rats. This model eliminates possible opioid abstinence that can occur with intermittent administration methods, and minimizes animal stress associated with other methods of handling and injecting, which could lead to associative learning and memory (Granados-Soto, et al. (2000) Pain 85:395-404). Accordingly, individual groups of eight rats were prepared with i.t. catheters and allowed to recover for 5 to 7 days to ensure no motor deficiency due to catheter implantation. Rats then were implanted with morphine or placebo pellets subcutaneously. Morphine antinociceptive tests were performed before pelleting (day 0), and days 1, 3, 5, and 7 after pelleting. Dose-response curves of morphine (i.t. bolus injections) were constructed in rats receiving placebo or morphine pelleted. A significant decrease in % MPE at given doses from the prepelleting baseline values signified the development of morphine antinociceptive tolerance. CaMKII expression and activity (pCaMKII) in lumbar segments of spinal cord was determined on days 0, 1, 3, 5, and 7 relative to morphine pelleting in order to establish the time course, which was compared to the onset of opioid antinociceptive tolerance. β-Actin was used as the internal control for quantitative comparison between samples. The results of this analysis indicated that the average increase of CaMKII activity from two pairs of animals was 250%. Spinal CaMKII activity, as measured by the active CaMKII (pCaMKII) content, was increased in rats made tolerant to morphine. Therefore, these results demonstrated the important of spinal CaMKII in opioid tolerance.

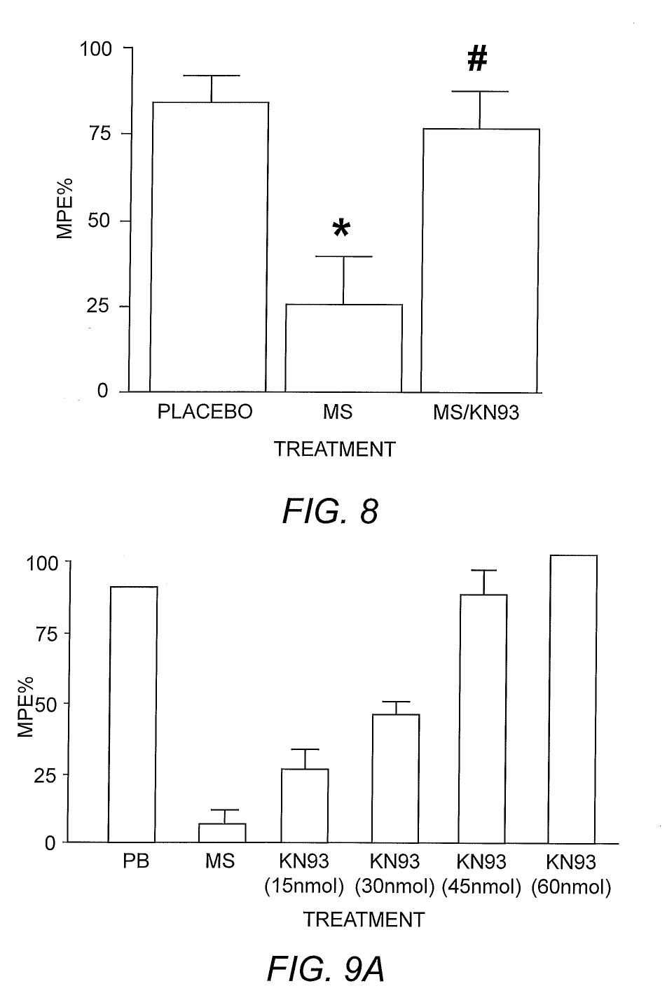

Accordingly, tests were performed to determine whether spinally applied KN93, a CaMKII inhibitor, disrupts morphine antinociceptive tolerance in rats. Individual groups of eight rats were implanted with i.t. catheters and allowed to recover for 5 to 7 days. Subsequently, the rats were implanted subcutaneously (s.c.) with either morphine (two 75 mg pellets/rat, NIDA) or placebo pellets. Baseline nociception and morphine antinociceptive effects were tested prior to pelleting. Five days after pelleting, the rats were tested for latencies in tail-flick test using 52° C. warm water before and 30 minutes after i.t. acute injection of morphine (10 μg in 5 μl saline). FIG. 8 shows that chronic morphine treatment produced antinociceptive tolerance to i.t. morphine (p<0.05). The reduced morphine antinociceptive effect in morphine-pelleted rats (MS) was reversed by i.t. administration of KN93 (15 nmol/5 pl saline) 15 minutes before the i.t. morphine (i.e., 45 minutes before tail-flick testing; MS/KN93) (*p<0.05 compared to Placebo group; #p<0.05 compared to MS group). Morphine had a significantly reduced antinociceptive effect in morphine-pelleted animals compared to the effect of morphine in rats received placebo pellets (FIG. 8), or prepelleting baseline.

These results indicate that the rats were morphine tolerant and morphine antinociceptive tolerance was blocked by administration of KN93 (15 nmol in 5:1 saline, i.t. injection) 15 minutes before acute challenge of morphine (FIG. 8). KN93 alone did not alter basal nociception, nor did KN93 affect morphine-antinociception in naïve rats.

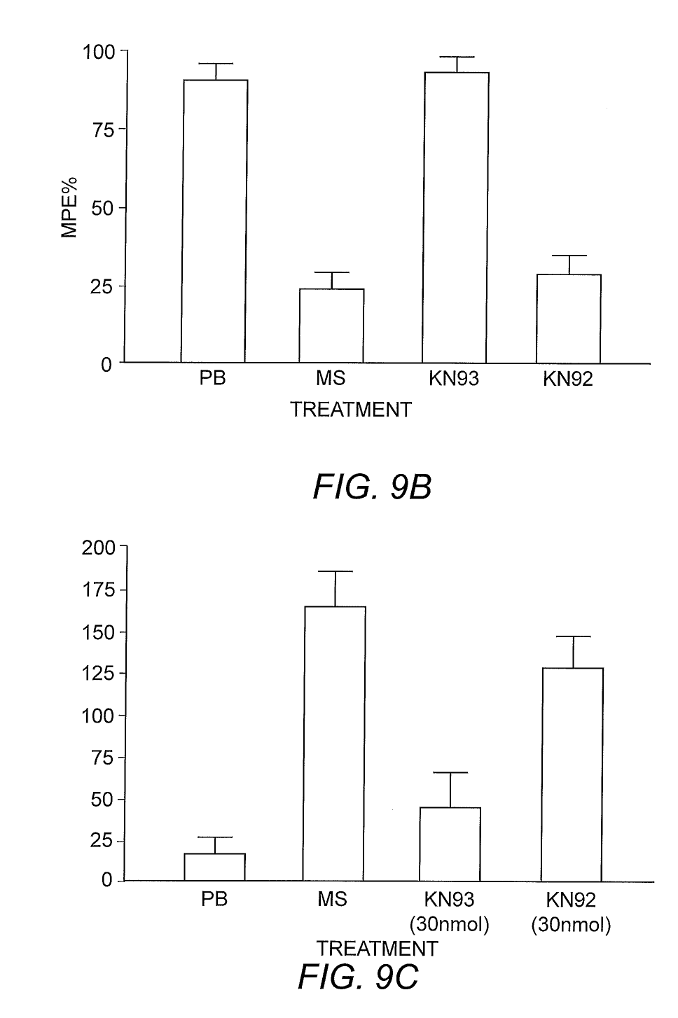

Additional in vivo tests corroborated initial test results showing that administration of a CaMKII inhibitor reduces or eliminates opioid tolerance and dependence. For example, in a mouse model of opioid tolerance due to chronic treatment with morphine (s.c. implantation of 75 mg controlled-release pellet, for up to seven days), morphine (given i.v., i.th., or perperally) produced significantly reduced antinociceptive effects (FIG. 9A). FIG. 9A shows that KN93 dose-dependently reverses established opioid tolerance in a chronic model of opioid tolerance. Administration of a CaMKII inhibitor, i.e., KN93, effectively reversed the established tolerance to opioids. The effect of KN93 was dose-dependent. The same chronic treatment with morphine also produced drug dependence in mice, which was also reversed by acute administration of CaMKII inhibitors.

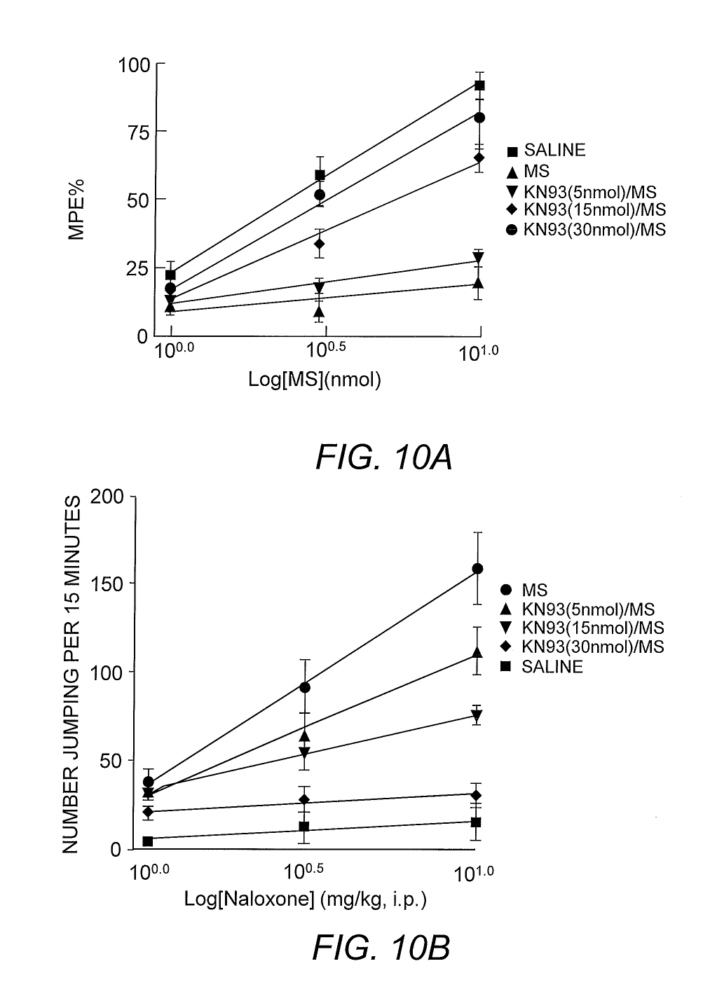

Acute tolerance and dependence model is a method commonly used by researchers. In this model, opioid tolerance and dependence are induced by a single s.c. injection of morphine (100 mg/kg). KN93 prevented the development of opioid tolerance and dependence when administered simultaneously with morphine (FIGS. 9B and 9C). FIG. 9B shows that KN93 prevents opioid tolerance in an acute model of opioid tolerance. A close analogue, but inactive form KN92, does not affect opioid tolerance. FIG. 9C shows that KN93 dose-dependently prevents opioid dependence in an acute model of opioid dependence. In addition, the CaMKII inhibitor KN93 also was effective in reversing an already-established tolerance or dependence in the model (FIGS. 10A and 10B). FIG. 10A shows that KN93 dose-dependently reverses established opioid tolerance in an acute model of opioid tolerance. FIG. 10B shows that KN93 dose-dependently reverses established opioid dependence in an acute model of opioid dependence. All effects are dose-dependent on the magnitude of inhibition of CaMKII.

The test results clearly demonstrate that a CaMKII inhibitor, e.g., KN93, does not affect morphine-induced analgesia. This is an important clinical finding because administration of a CaMKII inhibitor combined with administration of morphine does not interfere with the acute therapy of opiate analgesics and does not affect pharmacological actions of morphine.

On the basis of these test results, a CaMKII inhibitor reduces the dose of morphine and still produces same degree of analgesic action of morphine in opioid-tolerant state compared to a higher dose of morphine used alone. Lowering the dose of morphine can significantly reduce the addiction potential of morphine in patients.

### Example 5

### Trifluoperizine Disrupts Antinociceptive Tolerance

Male ICR mice (20-25 grams; Harlan, Indianapolis, Ind.) used in these studies were housed under a 12:12 hour light/dark cycle with access to food and water ad libitum. Trifluoperazine (Sigma, St. Louis, Mo.) and morphine sulfate (Abbott Laboratories, North Chicago, Ill.) were prepared in normal saline. For each experiment, differences among all groups were first analyzed by ANOVA. When a statistical significance was detected, Student's t-test was used to determine the statistical difference between a testing group and its corresponding control group. Statistical significance was established at 95%.

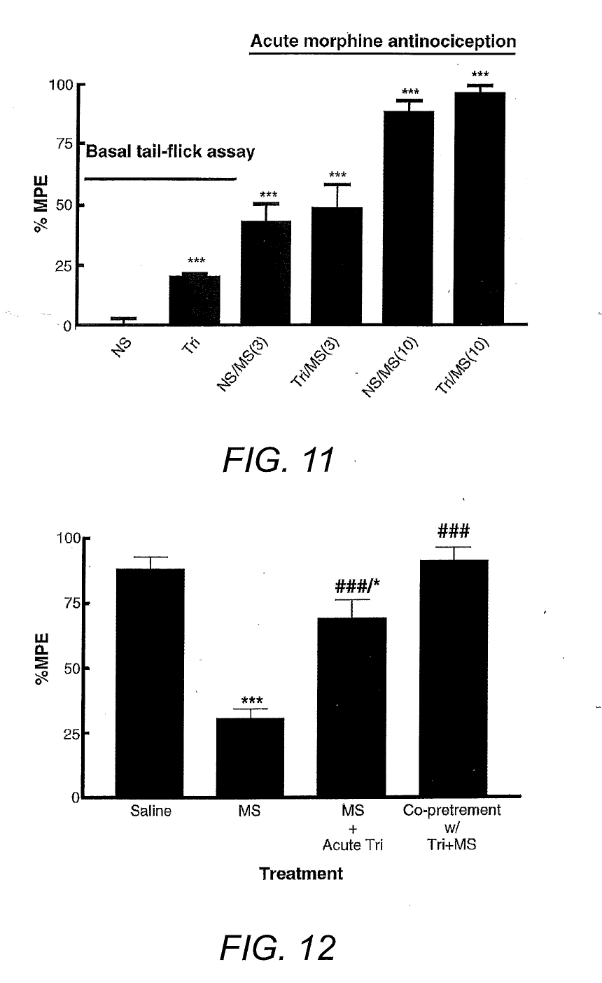

It was first determined whether trifluoperazine itself produced antinociception or affected the antinociceptive effect of morphine. Trifluoperazine (0.5 mg/kg, i.p.) produced antinociception (20.4±1.2% MPE) in a warm water (52° C.) tail-flick test when given to naïve mice (FIG. 11).

To test antinociception, the latencies of tail flick responses were measured before and 30 minutes after the administration of morphine (s.c.). A cut-off of 12 seconds was applied to prevent tissue damage. Results were presented in “% MPE” (maximal possible effect) as defined by the formula: % MPE=100×(test-control)/(cut-off-control). When trifluoperazine was given 30 minutes before the administration of morphine, it did not enhance morphine (10 mg/kg, s.c.) antinociceptive response. To rule out a potential ceiling effect, another experiment was performed using a lower dose of morphine (3 mg/kg, s.c.). Similarly, trifluoperazine did not alter the antinociceptive effect of morphine (3 mg/kg).

To induce tolerance, mice were treated with morphine sulfate (100 mg/kg, s.c.) using conventional methods (Bilsky et al. (1996) supra). Control mice received an equal volume of saline (s.c.). Mice were tested for the presence of opioid tolerance, 5 hours later, by monitoring the antinociception produced by a test dose of morphine (10 mg/kg, s.c.). To test the effect of trifluoperazine on morphine tolerance, mice were given trifluoperazine (0.5 mg/kg, i.p.) 4 hours after the treatment with morphine (100 mg/kg, s.c.) or saline (i.e., 30 minutes before the test dose of morphine). Morphine (10 mg/kg, s.c.) produced 88.2±4.7% MPE in saline-treated mice (FIG. 11), which was not different from its antinociceptive effect in naïve untreated mice. However, the same test dose of morphine produced significantly lower antinociception (30.7±3.6% MPE, p<0.001) in morphine (100 mg/kg)-treated mice, indicative of the development of morphine antinociceptive tolerance (FIG. 12). When trifluoperazine was given 30 minutes before the test dose of morphine, morphine antinociceptive tolerance was completely abolished in morphine (100 mg/kg)-treated (i.e., tolerant) mice (p<0.001 compared with morphine group; not significantly different from saline group; FIG. 12). These data indicated that the trifluoperazine reversed the established acute morphine antinociceptive tolerance.

It was further determined if pretreatment with trifluoperazine could prevent the development of morphine antinociceptive tolerance. In these experiments, mice were injected with trifluoperazine (0.5 mg/kg, i.p.) immediately before the administration of morphine (100 mg/kg, s.c.). Compared with mice that received morphine alone, mice co-treated with trifluoperazine and morphine showed significantly reduced antinociceptive tolerance to morphine (p<0.001; FIG. 11). Co-treated mice still exhibited some tolerance when compared to those that received saline (p<0.05). These data indicated that trifluoperazine was also effective in preventing the development of morphine antinociceptive tolerance. The incomplete prevention of morphine tolerance may be due to relatively short duration of action of trifluoperazine, since its peak plasma level occurs less than 3 hours following oral administration in humans (Midha, et al. (1983) Br. J. clin. Pharmacol. 15:380-382).

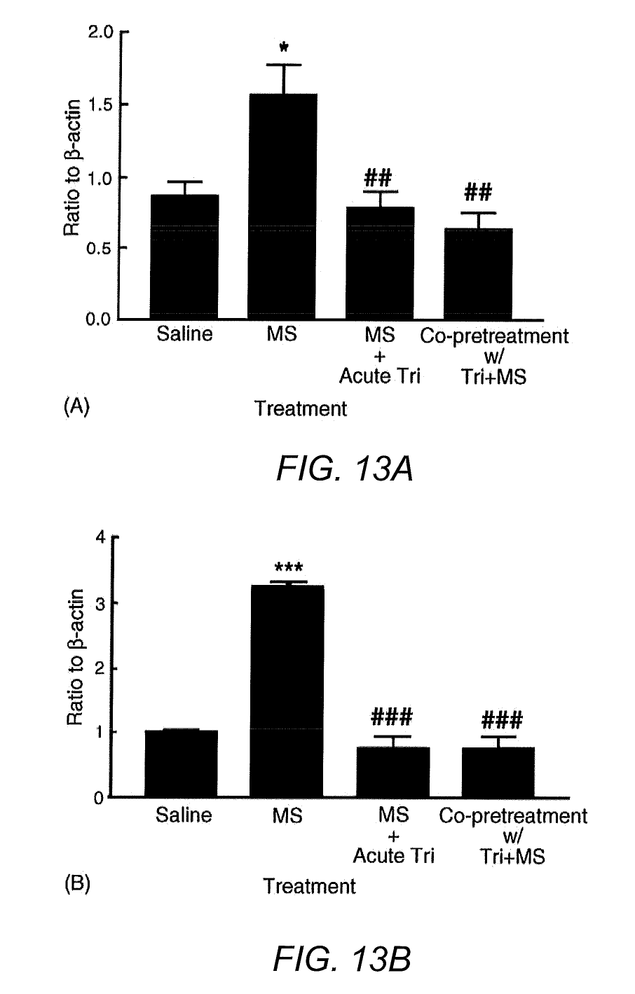

Since trifluoperazine did not alter acute morphine antinociception (FIG. 11), its effect on morphine tolerance could not be due to directly enhancing acute morphine antinociception. To correlate the behavioral effect of trifluoperazine with its cellular inhibitory effect on CaMKII activity, the CaMKII activity in mice treated with morphine and/or trifluoperazine was examined. Brain and spinal CaMKII activities were determined using western blot analysis (Wang, et al. (1999) J. Biol. Chem. 274:22081-22088; Wang, et al. (2001) J. Neurochem. 21:1779-1786). Solubilized tissue samples were subjected to 10% polyacrylamide gel electrophoresis and transferred onto PVDF membranes, which were then probed with a rabbit antibody recognizing the activated form of CaMKII (anti-pCaMKII antibody, 1/1000; Promega, Madison, Wis.), followed by the incubation with HRP-conjugated donkey anti-rabbit secondary antibody ( 1/1000; Amersham, Piscataway, N.J.). The membranes were developed using an enhanced chemiluminescence (ECL) detection system (Amersham). ECL-signals were captured by a ChemiDoc imaging system and analyzed using Quantity One program (BIO-RAD, Hercules, Calif.). The membranes were then stripped and re-probed with a mouse anti-β-actin antibody ( 1/10,000; Sigma), then an anti-mouse HRP-conjugated secondary antibody ( 1/10,000; Amersham), and developed as above. CaMKII activity was significantly increased in the brain (81% increase, p<0.05) and spinal cord (222% increase, p<0.001) of mice tolerant to morphine compared with saline-treated mice (FIG. 13). The enhanced CaMKII activity was completely abolished in mice pretreated with both morphine and trifluoperazine or acutely treated with trifluoperazine (FIG. 13).

The results of this analysis indicated that trifluoperazine effectively reversed and significantly prevented the development of acute antinociceptive tolerance to morphine. Since higher doses of trifluoperazine are required to produce antipsychotic effects, it is expected that CaMKII activity can be inhibited by trifluoperazine at the doses that are used to treat psychotic disorders in humans.

### Example 6

### Haloperidol Disrupts Opioid Antinociceptive Tolerance and Dependence

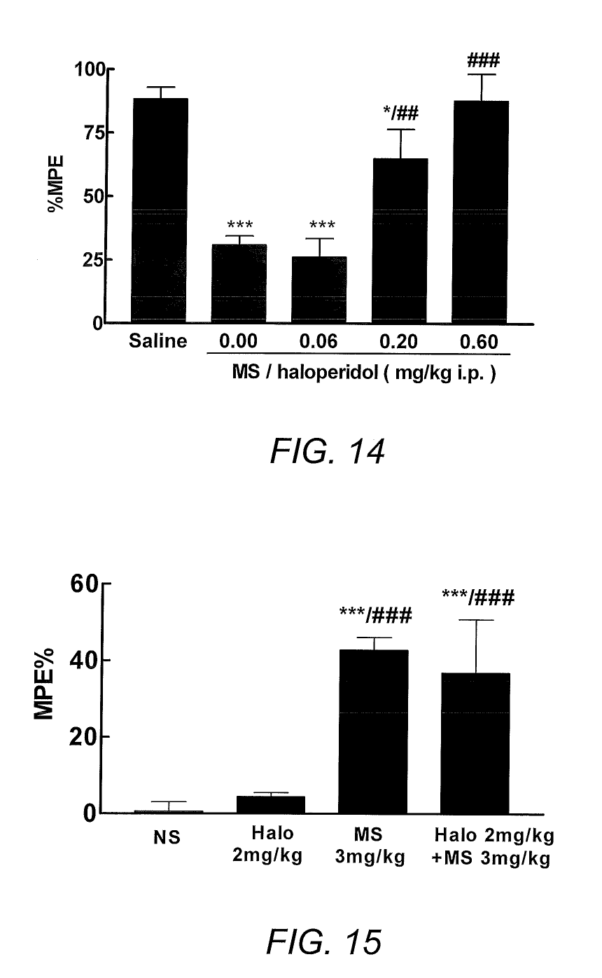

To demonstrate the effects of haloperidol, it was first determined whether acute treatment of haloperidol was able to reverse the established morphine tolerance and dependence. Acute morphine tolerance and dependence were established 2 to 6 hours after morphine injection (100 mg/kg s.c.) (Bilsky, et al. (1996) supra). The results of this analysis indicated that morphine produced significant reduced antinociception (30.73±3.57% MPE, p<0.001) in tolerant mice compared to saline-pretreated mice (88.16±3.57% MPE). Haloperidol (0.06, 0.20, 0.60 mg/kg i.p.) administered 1 hour before antinociceptive test reversed the established morphine tolerance. This reversal was dose-dependent. High dose haloperidol (0.60 mg/kg i.p.) completely reversed morphine tolerance (87.65±10.74% MPE p<0.001) while low dose haloperidol (0.06 mg/kg i.p.) was less effective (26.13±7.23% MPE) (FIG. 14).

To investigate whether haloperidol itself produced antinociception or affected the antinociceptive effect of morphine, haloperidol (2.0 mg/kg i.p.) was given alone to naïve mice and co-administrated with low dose of morphine (3.0 mg/kg s.c. to reduce the sailing effect). At this dose, haloperidol itself did not produce antinociception (4.43±2.10% MPE) in a warm water (52° C.) tail-flick test and it did not alter the antinociceptive effect of morphine (FIG. 15).

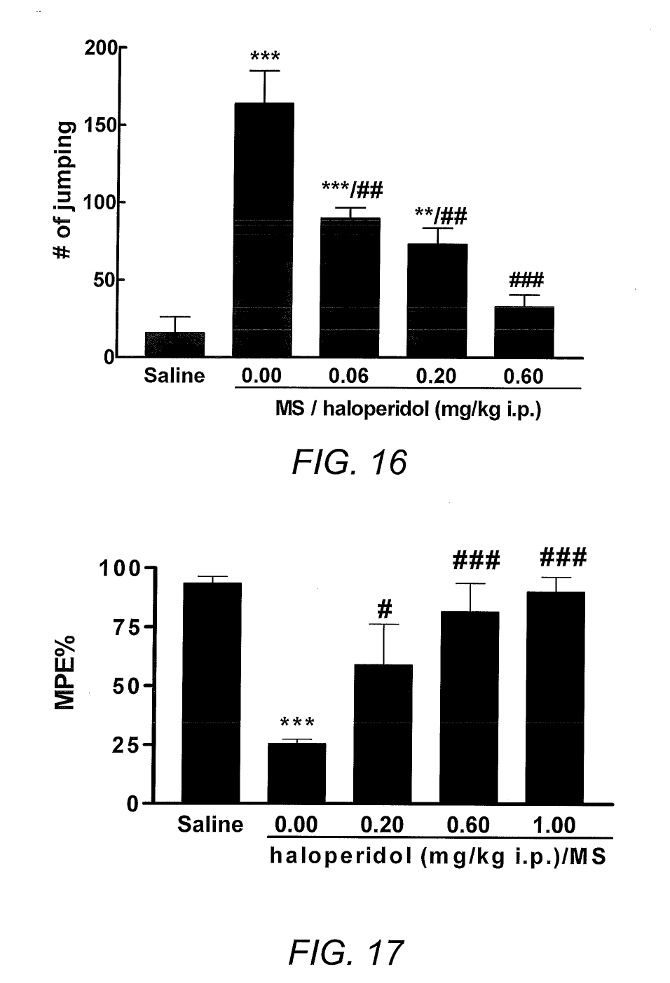

To test the effect of haloperidol on acute morphine dependence, mice were treated with morphine (100 mg/kg i.p.) so that dependence on morphine developed in 2 to 6 hours (Bilsky, et al. (1996) supra). Naloxone (10 mg/kg i.p.) withdraw dumping was evaluated 5 hours after morphine injection. Haloperidol (0.06, 0.20, 0.60 mg/kg i.p.) was given 30 minutes before naloxone injection. The results of this analysis indicated that haloperidol could dose-dependently attenuate naloxone-induced withdraw jumping. At the highest dose (0.60 mg/kg), haloperidol was able to completely suppress the withdraw jumping. Lower doses (0.06 and 0.20 mg/kg) were also able to significantly attenuate withdraw jumping (p<0.001) (FIG. 16).

It was subsequently investigated whether haloperidol could prevent the development of opioid tolerance. In these studies, haloperidol (1.0, 0.6, 0.2 mg/kg s.c.) was injected 30 minutes before the injection of morphine (100 mg/kg s.c.). Acute morphine tolerance was established 2 to 6 hours after morphine injection. Tolerant mice showed a significant decrease in morphine antinociception (25.54±3.98, % MPE) produced by the second test dose of morphine (10 mg/kg s.c.) compared with saline pretreated mice (93.57±6.44, % MPE). In all haloperidol pretreatment groups, morphine-induced tolerance was attenuated. In the higher dose haloperidol pretreatment groups (1.0 and 0.6 mg/kg s.c.), morphine-induced tolerance was absent (90.19±14.05% MPE, p<0.001; 81.62±26.96% MPE, p<0.001 respectively compared with morphine tolerance mice; not significantly different from saline group). In the lowest dose haloperidol (0.2 mg/kg i.p.) pretreatment group, the morphine-induced tolerance was partially attenuated (59.01±38.65% MPE, p<0.05) (FIG. 17).

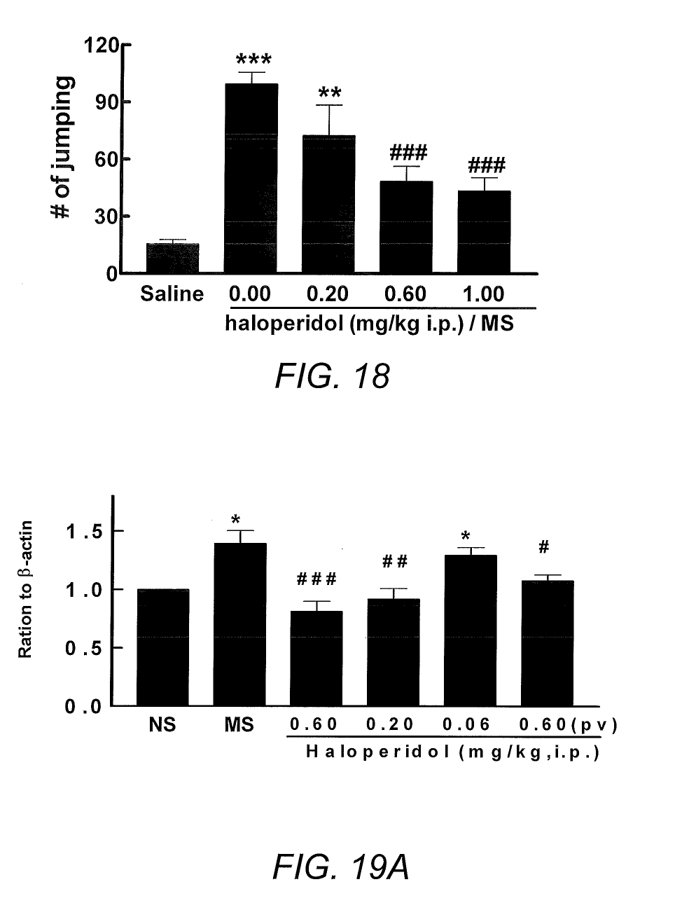

Prevention of the development of opioid dependence was also analyzed. Mice were treated with haloperidol (1.0, 0.6, 0.2 mg/kg i.p.) and morphine (100 mg/kg s.c.) as indicated herein. Naloxone (10 mg/kg i.p.) was given 6 hours after morphine injection. Withdraw dumping in the first 15 minutes after naloxone administration was counted for evaluating the effect on opioid dependence. The results of this analysis indicated that haloperidol could prevent development of the opioid dependence in a dose-dependent manner. Higher dose haloperidol (1.0 and 0.6 mg/kg i.p.) significantly prevented the development of opioid dependence, whereas the lowest dose (0.2 mg/kg i.p.) had a slight effect (FIG. 18).

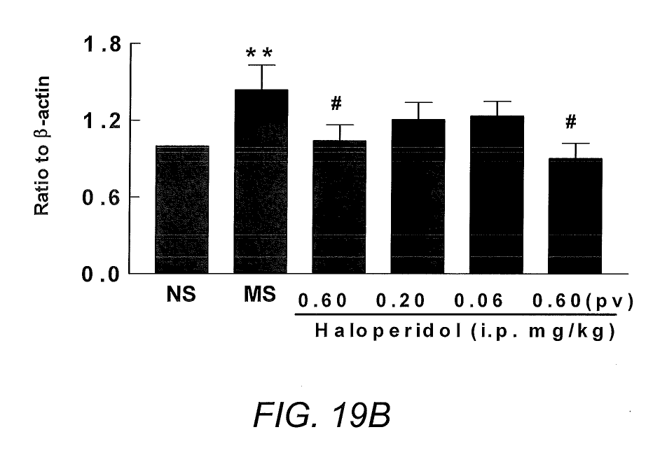

To explain the cellular mechanism of these behavioral effects, pCaMKII activity in mice treated with morphine and/or different dose of haloperidol was analyzed. Mice in different groups received morphine (100 mg/kg s.c.). Control group was given the same amount of saline instead. Haloperidol was administrated in a different manner. For the prevention experiment, haloperidol (0.6 mg/kg i.p.) was given 0.5 hours before the morphine injection. For the reversion test, haloperidol (0.60, 0.20, 0.06 mg/kg i.p.) was given 5 hours after the morphine injection. Mice were sacrificed 6 hours after the morphine injection. Brain cortexes and spinal cords were taken for western blot analyses. As shown in FIGS. 19A and 19B, pCaMKII activities were significantly increased in morphine group (p<0.5, p<0.01 respectively, compared with saline group) in both brains and spinal cords. Haloperidol dose-dependently attenuated the over-expression of pCaMKII activities in reversion experiments. Higher doses of haloperidol (0.6 and 0.2 mg/kg i.p.) significantly decreased pCaMKII activities in brain (p<0.001, p<0.01 respectively, compared with morphine group), while only the highest dose (0.6 mg/kg i.p) had a statistically significant effect in spinal cord (p<0.05 compared with morphine group). Pretreatment with haloperidol (0.6 mg/kg i.p.) completely prevented increases in pCaMKII activity in both brain and spinal cord.

### Example 7

### CaMKIIα is Required for the Initiation and Maintenance of Opioid-Induced Hyperalgesia

Chronic morphine Exposure Induced Mechanical Allodynia and Thermal Hyperalgesia in Mice.

Repeated s.c. morphine administration and s.c. morphine pellet implantation are two commonly used OIH models in mice. Four days of s.c. morphine administration by intermittent injections significantly increased mechanical and thermal sensitivities compared with saline treated mice. Mechanical allodynia and thermal hyperalgesia were detectable on day 5 and lasted for about 2 weeks before recovery (p<0.001 compared with saline control, n=5).

Continuous morphine exposure using pellet implantation also induced OIH. Mice were implanted subcutaneously with morphine pellets (75 mg/pellet) or placebo pellet and mechanical and thermal sensitivities were measured daily for 15 days. Morphine implantation initially produced antinociception in both mechanical (p<0.001, compared with the placebo group, n=5) and thermal sensitivity tests (p<0.05, compared with the placebo group, n=5). This was followed by a decrease in paw withdrawal threshold and latency. Mechanical allodynia was developed on day 6 and lasted for 7 days (p<0.001, compared with placebo group, n=5). Thermal hyperalgesia was also observed from day 5 to day 9 (p<0.001, compared with placebo group, n=5) after morphine implantation. Comparing the two OIH models, repeated intermittent morphine administration led to longer lasting and more robust mechanical allodynia and thermal hyperalgesia in ICR mice. Therefore, this model was used for the CaMKII intervention studies.

CaMKII Inhibition by KN93 Reversed Morphine-Induced Hyperalgesia.

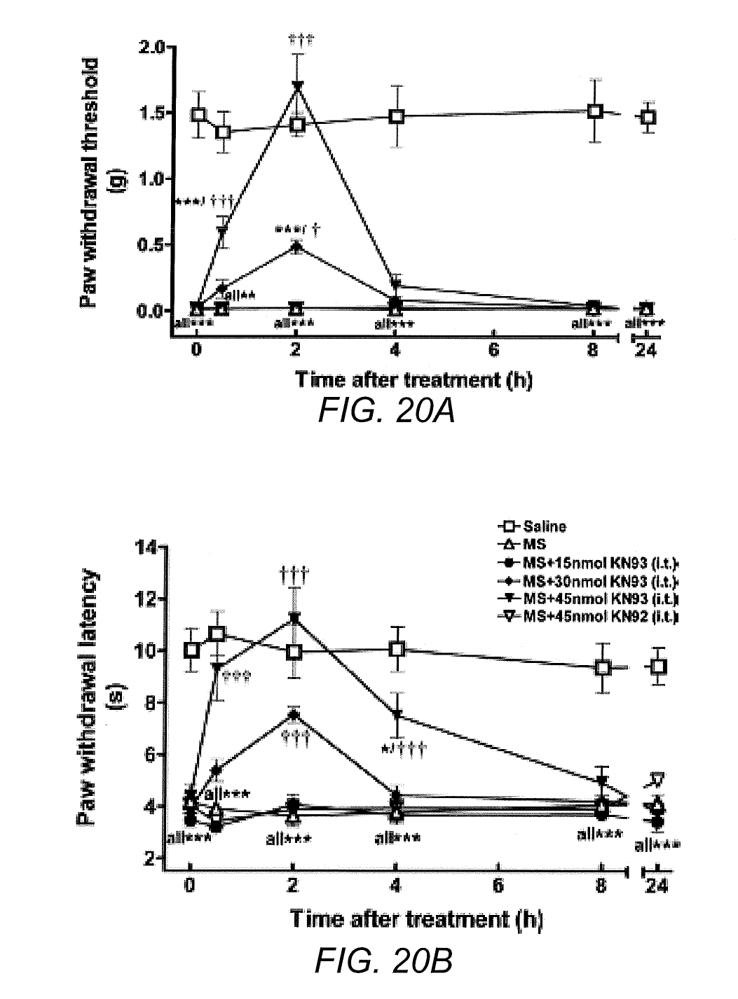

To investigate the role of CaMKII in OIH, KN93 was employed as was KN92 as a negative control. Both mechanical allodynia and thermal hyperalgesia were significantly developed on day 5. At that point, mice were treated with KN93 (15-45 nmol, i.t.) or KN92 (45 nmol, i.t.) and mechanical and thermal sensitivities were monitored for 24 hours. Opioid-induced hyperalgesia was found to be attenuated by KN93 in a dose- and time-dependent fashion. At the highest dose, KN93 (45 nmol) completely reversed allodynia and hyperalgesia. Its anti-allodynic/anti-hyperalgesic effect appeared 30 minutes after KN93 administration and peaked at 2 hours (FIG. 20). The anti-allodynic/anti-hyperalgesic action of KN93 diminished at 8 hours post-administration. At lower doses, KN93 (30 nmol, i.t.) partially suppressed allodynia and hyperalgesia, while KN93 at 15 nmol did not affect either thermal or mechanical sensitivity at any time point tested. At the peak-effect time (2 hours), ED₅₀ values were estimated to be 26.1±3.4 nmol (anti-allodynic) and 17.8±3.0 nmol (anti-hyperalgesic). By contrast, KN92 (45 nmol, i.t.) did not alter the pain threshold at any time point tested, which strongly supported a CaMKII-mediated effort exhibited by KN93. To rule out a potential negative effect of KN93 on locomotor coordination that can be a confounding factor in data interpretation, the effect of KN93 was further tested on locomotor activity in a rotarod test. Administration of KN93 (45 nmol, i.t.) or KN92 (45 nmol, i.t.) to naïve mice did not produce significant changes to the locomotor coordination in the rotarod test (p>0.05, compared with saline treated group, n=5). These data were in agreement with the observation that KN93 (45 nmol, i.t.) does not change mechanical and thermal sensitivities in naïve mice.

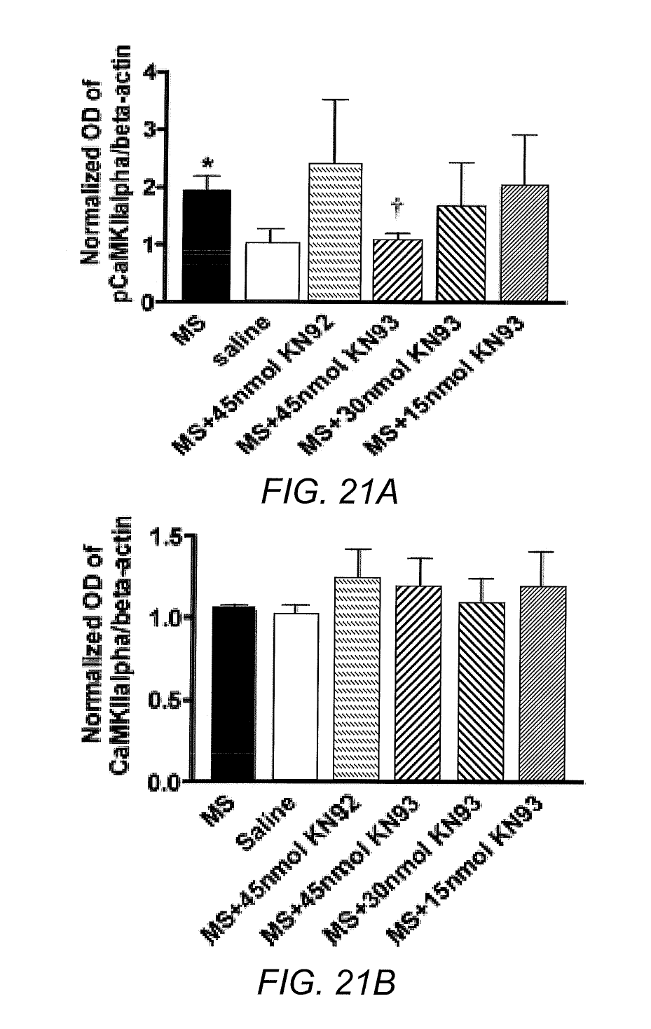

In order to correlate behavioral effects with biochemical changes, CaMKIIα activity was determined by analyzing the degree of autophosphorylation (pCaMKIIα) (Fang, et al. (2002) supra; Xu & Huang (2004) Proc. Natl. Acad. Sci. USA 101:11868-11873). Compared with the saline control, repeated morphine administration significantly increased spinal pCaMKIIα (FIG. 21A; p<0.05, n=4), without significantly altering its expression (FIG. 21B; p>0.05, n=4). KN93, at the highest dose used (45 nmol, 1 hour), significantly reversed the morphine-induced activation of CaMKIIα (p<0.05, compared with morphine group, n=4). In contrast, KN92 (45 nmol) did not change morphine-induced pCaMKIIα. Activation of spinal CaMKIIα was also examined using immunohistochemical approach. After chronic morphine exposure, increased pCaMKIIα immunoreactivity was found mostly in the superficial laminae of the spinal dorsal horn compared with the saline-treated mice. Some pCaMKIIα immunoreactivity was also found in the neuropils, consistent with previous reports (Song, et al. (2009) Brain Res. 1271:114-120; Liang, et al. (2004) Neuroscience 123:769-775; Fujiyoshi, et al. (2007) J. Neurosci. 27:11991-11998). However, the enhanced pCaMKIIα immunostaining was significantly suppressed by KN93 (45 nmol, i.t.). These data indicated that chronic morphine induced mechanical allodynia and thermal hyperalgesia, and their reversal by KN93, are biochemically correlated with morphine-induced CaMKIIα activation and subsequent inactivation in the presence of KN93, but not KN92.

Small Interfering RNA (siRNA)-Mediated Knockdown of CaMKIIα and Reversal of Morphine-Induced Hyperalgesia.

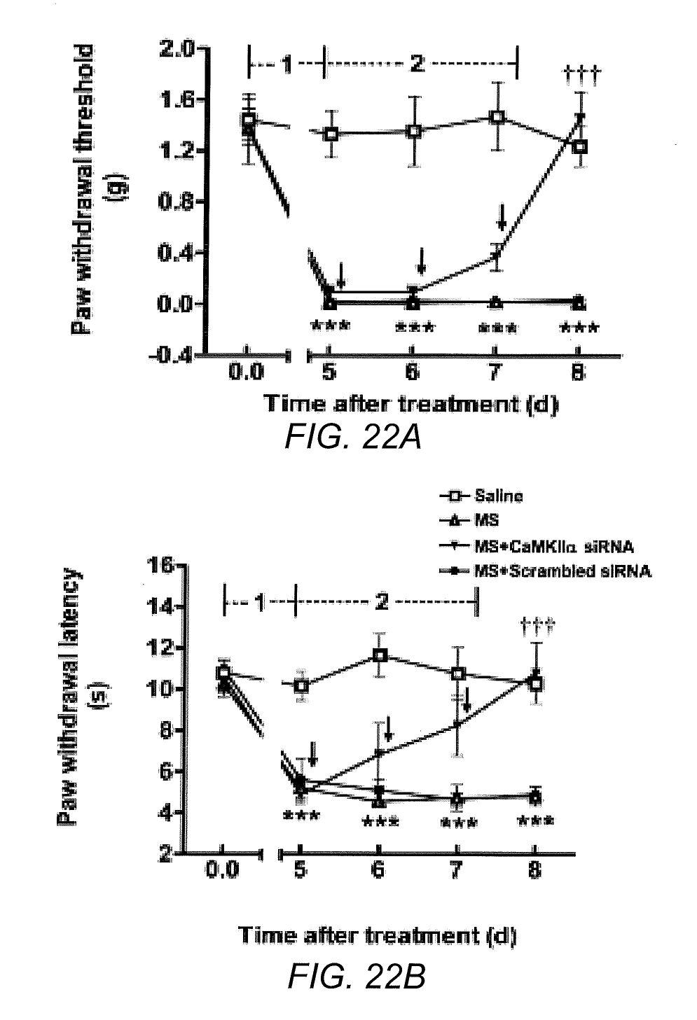

To further investigate the specific isoform of CaMKII involved in OIH, siRNA targeting CaMKIIα (Zayzafoon, et al. (2005) supra) was applied in order to knock down the expression of CaMKIIα in the spinal cord. Mice received repeated morphine administration in phase 1 (FIG. 22) and developed mechanical allodynia and thermal hyperalgesia 18 hours after the final morphine injection. In phase 2, mice received intrathecal administration of CaMKIIα siRNA or scrambled siRNA (2 μg/injection, twice per day for 3 consecutive days) and the sensitivities to mechanical and thermal stimuli were measured daily. Treatment with CaMKIIα siRNA gradually attenuated morphine-induced mechanical allodynia (FIG. 22A) and thermal hyperalgesia (FIG. 22B). After 3 days of treatment, OIH was completely reversed (FIG. 22, p<0.001, compared with morphine treated group, n=5). Correlating with the behavioral effect, pCaMKIIα immunostaining in the spinal dorsal horn was significantly suppressed. In contrast, scrambled siRNA did not alter OIH (FIG. 22) or morphine-induced pCaMKIIα immunoreactivity.

Absence of Morphine-Induced Hyperalgesia in CaMKIIα^{T286A} Point Mutation Mice.

To investigate whether functional CaMKIIα was also required for the development of OIH, CaMKIIα^{T286A} mice were analyzed. These mice were engineered to carry a T286A point mutation, lacking the ability to undergo CaMKIIα autophosphorylation and further activation (Giese, et al. (1998) supra). It was investigated whether inactivation of CaMKIIα by T286A point mutation affected morphine-induced hyperalgesia. Separate groups of 6 male CaMKIIα^{T286A} mutant and littermate wild-type mice received repeated injections of morphine every 12 hours for 4 days using the same treatment schedule as described herein. Baseline pain thresholds were not significantly different between wild-type and mutant mice. The wild-type mice exhibited mechanical allodynia and thermal hyperalgesia on day 5, indicative of the presence of OIH (p<0.01, compared with baseline, n=6). However, mechanical allodynia and thermal hyperalgesia were not detected in morphine-treated CaMKIIa^{T286A} mutant mice. These data indicated that CaMKIIα is required for the initiation of OIH.

These data implicate CaMKIIα as a cellular mechanism leading to and maintaining opioid-induced hyperalgesia. This evidence highlights an important role for CaMKII in morphine tolerance and dependence and indicate that targeting CaMKII may be useful for the prevention and treatment of OIH.

What is claimed is:

1. A method for preventing or treating pain comprising administering to a subject in need of treatment an effective amount of a trifluoperazine thereby preventing or treating the subject's pain.

2. The method of claim 1, further comprising administering an effective amount of an opiate analgesic.

3. The method of claim 2, wherein the trifluoperazine and opiate analgesic are administered simultaneously.

4. The method of claim 2, wherein the trifluoperazine and opiate analgesic are administered sequentially.

5. The method of claim 2, wherein the opiate analgesic is an opium alkaloid, a semisynthetic opiate analgesic, or a mixture thereof.

6. The method of claim 1, wherein the pain is acute or chronic pain.

7. The method of claim 6, wherein the chronic pain is cancer pain, post-traumatic pain, post-operative pain, neuropathic pain, inflammatory pain or pain associated with a myocardial infarction.
